(19) United States
(12) Patent Application Publication
(10) Pub. No.: US 2025/0333733 A1
(43) Pub. Date: Oct. 30, 2025

(54) NOVEL TARGETS AGAINST OVARIAN CANCER

(71) Applicant: UNIVERSITY OF PUERTO RICO, San Juan, PR (US)

(72) Inventors: Pablo VIVAS-MEJIA, San Juan, PR (US); Ricardo NORIEGA, San Juan, PR (US)

(21) Appl. No.: 18/866,512

(22) PCT Filed: May 19, 2023
(86) PCT No.: PCT/US2023/067238

§ 371 Date: Nov. 15, 2024

Related U.S. Application Data
(60) Provisional application No. 63/343,974, filed on May 19, 2022.

(51) Int. Cl. C12N 15/113; A61K 9/1271; A61P 35/00

(52) U.S. Cl. CPC C12N 15/113; A61K 9/1271; A61P 35/00

(57) ABSTRACT

The present disclosure provides a method of treating ovarian cancer comprising administering an siRNA against CASC10, wherein the CASC10 gene expression is reduced following administration in ovarian cancer patients.

### CROSS-REFERENCE TO RELATED APPLICATIONS

This application claims benefit under 35 U.S.C. § 119(e) of U.S. Provisional Application No. 63/343,974 filed May 19, 2022. The above listed application is incorporated by reference herein in its entirety for all purposes.

### STATEMENT REGARDING FEDERALLY SPONSORED RESEARCH

This invention was made with government support under award numbers U54MD007600 awarded by the National Institute of Health. The government has certain rights in the invention.

### REFERENCE TO AN ELECTRONIC SEQUENCE LISTING

The instant application contains a Sequence Listing which has been submitted electronically as a text file in ASCII format and is hereby incorporated by reference in its entirety. The name of the ASCII text file is “22-10882-WO_Sequence-Listing_ST26_FINAL.txt”, was created on May 16, 2022, and is 492 kilobytes in size.

### FIELD OF THE INVENTION

The present disclosure relates to the treatment of ovarian cancer. More specifically, the disclosure relates to silencing of genes, such as CASC10, associated with ovarian cancer. The result is an unexpected decrease in tumor volume.

### BACKGROUND

Ovarian cancer is a leading cause of death in women. Epithelial ovarian carcinoma (EOC) is the most common ovarian cancer type representing 90% of the malignancies [2]. High-grade serous ovarian cancer (HGSOC) represents 70% of all EOCs [3]. The standard line of treatment for ovarian cancer usually consists of cytoreductive surgery combined with chemotherapy with platinum (i.e., cisplatin) and/or taxane-based compounds [4]. Despite this, treatments for ovarian cancer are largely ineffective. While initial response rates are 60-80%, approximately 70% of HGSOC develop a cisplatin-resistant-fatal disease [5]. The major contributors to the cisplatin resistance of ovarian cancer cells have not been fully identified.

For instance, despite initial responses to first-line treatment with platinum and taxane-based combination chemotherapy, most high-grade serous ovarian carcinoma (HGSOC) patients will relapse and eventually develop a cisplatin-resistant fatal disease. Due to the lethality of this disease, there is an urgent need to develop better-targeted therapies against HGSOC. Gene targeting of both primary and downstream genes provide a possible mechanism for treating HGSOC, by targeting cell survival, apoptosis, cell cycle progression, and tumor growth using an ovarian cancer mouse model. There is a need in the art for methods of reducing tumor growth and metastasis using gene silencing and liposomal formulations. Such methods are disclosed herein.

### SUMMARY

The disclosure provides a method of treating ovarian cancer. The disclosure also provides liposomes, pharmaceuticals and kits for siRNA knockdown of CASC10.

Specific embodiments of the disclosure will become evident from the following more detailed description and the claims.

As described below, in a first aspect is a method of treating cancer in a subject in need thereof, comprising administering an siRNA against one or more target genes SACS, CASC10, EMP1, GAS1, SLC6A15, GALNT13, ATP11B, and PDLIM3 resulting in reduced target gene expression following siRNA administration in cancer patients. In one aspect the SIRNA is CASC10 and after administration CASC10 gene expression is reduced in cancer cells in a subject in need thereof. In another aspect the cancer is ovarian cancer. In another aspect the siRNA is packaged inside a liposome.

In another aspect siRNA administration upregulates one or more of RTN4R, KIAA0754, PYM1, CNN1, and TGFBRAP1. In another aspect siRNA administration downregulates one or more of NUP43, FHL1, DHFR2, MIR1915HG, and NDUFA7.

In another embodiment is a liposome for use in treating ovarian cancer wherein the liposome contains one or more of CASC10, SACS, EMP1, GAS1, SLC6A15,GALNT13, ATP11B, or PDLIM3 siRNA. In one aspect the liposome siRNA is CASC10.

In one aspect is a pharmaceutical composition comprising the liposome wherein the SiRNA is CASC10.

In one aspect is a kit comprising the liposome containing an siRNA formulated for in vivo delivery of one or more siRNA comprising an siRNA mixed with DOPC in about a 1:2 to about a 1:20 ratio DSPE-PEG-2000 at a concentration of about 1% to about 10% mol/mol of DOPC; and cholesterol at a concentration of about 10% to about 40% w/w of DOPC.

In another aspect is a liposome formulation wherein the siRNA is one or more of CASC10, SACS, EMP1, GAS1, SLC6A15, GALNT13, ATP11B, or PDLIM3. In one aspect the targeted gene is CASC10. In one aspect the DSPE-PEGO-2000 concentration is about 5% mol/mol of DOPC. In another aspect the cholesterol concentration is 20%.

In another embodiment is a method for reducing cancer cell proliferation and/or invasion in an individual having ovarian cancer, the method comprising administering one or more of CASC10, SACS, EMP1, GAS1, SLC6A15, GALNT13, ATP11B, or PDLIM3 siRNA. In one aspect of the embodiment the siRNA is CASC10.

In one aspect the cancer is ovarian cancer and may be high-grade serous ovarian cancer (HGSOC) wherein the cells comprising the cancer may include VCAR or OVCAR3CIS positive cells.

In one aspect the administration of CASC10 reduces the number of ovarian cancer positive cell colonies by ≥50%. The positive colonies can include VCAR, OVCAR3CIS, and/or SKOV3ip1CIS cells.

In another aspect administration of CASC10 reduces ovarian cancer cell viability by ≥10%.

In another aspect administration of CASC10 induces apoptosis of cisplatin resistant cancer cells as assessed by increased capase-9 and capase-3 activity.

In another embodiment is a method of interfering with cancer cell cycle progression, the method comprising administering one or more siRNA targeting CASC10, SACS, EMP1, GAS1, SLC6A15, GALNT13, ATP11B, or PDLIM3.

In another embodiment is a method of reducing ovarian cancer tumor size by administering encapsulated siRNAs into DOPC-based liposomes, wherein the siRNA targets one or more of CASC10, SACS, EMP1, GAS1, SLC6A15, GALNT13, ATP11B, or PDLIM3.

In another embodiment is a method for treating an individual with cisplatin resistance ovarian cancer comprising administering to the individual CASC10 siRNA encapsulated in a liposome.

In one aspect the ovarian cancer is high-grade serous ovarian cancer (HGSOC) wherein the ovarian cancer is comprised of VCAR, OVCAR3CIS, and/or SKOV3ip1CIS positive cells.

### BRIEF DESCRIPTION OF THE DRAWINGS

FIGS. 1A-1E are schematics. FIG. 1A is a flowchart depicting the number of genes filtered in each step of RNA-seq. FIG. 1B is a volcano plot showing the global transcriptional changes in the pairs of cell lines. FIG. 1C is a heat map constructed with 414 differentially abundant transcripts from filter-3. FIG. 1D is an Ingenuity Pathway Analysis (IPA) of molecules involved in cell survival. FIG. 1E is an Ingenuity Pathway Analysis (IPA) of genes associated with vesicle trafficking, phosphorylation, and cGMP signaling.

FIG. 2A is Kaplan-Meier survival curves of overall survival (OS) and progression-free survival (PFSO by expression levels of (A) CASC10. (B) PDLIM3, (C) EMP1, and (D) ATP11B. FIG. 2B is a scatterplot of normalized expression of 45 differentially abundant transcripts between cisplatin Resistant and Sensitive OVCAR3CIS cells. FIG. 2C is a bar-graph representing siRNA screening for the 27 candidate genes by percent of clonogenicity. FIG. 2D is dot plot representing CASC10 expression levels in ovarian cancer tumor tissues (red) and normal tissues (black). FIG. 2E is a bar graph depicting fold change (relative to cisplatin-sensitive cells) of CASC10 expression.

FIG. 3A is a bar-graph depicting the RT-PCR fold change (relative to non-coding siRNA) following transfection in OVCAR3CIS cells. FIG. 3B is a bar-graph quantifying the colony formation assay in percent clonogenicity of OVCAR3CIS cells following transfection with siRNAs. FIG. 3C is a bar-graph depicting percent invasion of OVCAR3CIS cells following transfection with siRNAs. FIG. 3D is representative histology of the invasion assay. FIG. 3E is a viability plot of the siRNA transfected CIS resistant and CIS sensitive cells.

FIG. 4A is a bar-graph depicting Caspase-3 fluorometric activity in OVCAR3CIS cells 72 hours after transfection. FIG. 4B is depicting Western blot analysis of apoptotic-related proteins. FIG. 4C is a bar graph depicting the quantified band intensities of cell-death proteins between the transfected cells with non-coding and CASC10 SiRNAs. FIG. 4D is a bar graph depicting the quantified band intensities of BCL-2 protein levels between the non-coding and CASC10 siRNA transfected cells. FIG. 4E is a bar graph depicting the populations of cells at different stages of cell cycle arrest. FIG. 4F is a bar graph depicting the percentage of cells at each stage of cell cycle arrest by treatment group. FIG. 4G is a Western blot analysis of cell-cycle related proteins 48 hours after SIRNA infection. FIG. 4H is a bar graph depicting the quantified band intensities of cell-cycle related proteins between treatment groups. FIG. 4I is a bar-graph depicting the quantified band intensities of Cyclin E1 between the non-coding and CASC-10 siRNA transfected cells.

FIG. 5A is a bar graph comparing tumor weights (grams) between the treatment groups. FIG. 5B is a bar graph comparing the number of nodules between treatment groups. FIG. 5C is a representative photo showing extracted and weighed tumors in all groups. FIG. 5D is a bar-graph reporting the weight of mice (grams) in the different treatment groups at the end of therapy.

FIG. 6A is representative genomic information of CASC10. FIG. 6B is a representation of subcellular localization plots of CASC10. FIG. 6C is a Venn diagram showing 1400 RNA transcripts that are differentially abundant in OVCAR3CIS cells treated with NC-SIRNA and CASC10-siRNA (2). FIG. 6D is a gene ontology and KEGG analysis of the 20 most significantly enriched ontology clusters. FIG. 6E is a gene ontology and KEGG analysis of the 20 most significantly enriched ontology clusters regulated by transcription factors. FIG. 6F is an Ingenuity Pathway Analysis (IPA) following siRNA-mediated CASC10 knockdown.

FIG. 7 depicts Kaplan-Meier (KM) plots of overall survival (OS) and progression-free survival of ovarian cancer patients stratified by expression levels of 57 clinically relevant genes.

FIG. 8A is a bar graph depicting RT-PCR fold change (relative to non-coding SIRNA) following siRNA transfections in SKOV3ip1CIS cells. FIG. 8B is a bar graph quantifying the colony formation assay in percent clonogenicity of SKOV3ip1CIS cells following siRNA transfections. FIG. 8C is a bar graph depicting percent invasion of SKOV3ip1CIS cells following transfection with siRNAs. FIG. 8D is representative histology of the invasion assay. FIG. 8E depicts cell viability following siRNA transfections in SKOV3ip1CIS cells. FIG. 8F depicts cell viability following siRNA transfections in OVCAR3 cells. FIG. 8G depicts cell viability following siRNA transfections in SKOV3ip1 cells. FIG. 8H depicts cell invasion following siRNA transfection in SKOV3ip1 cells. FIG. 81 depicts cell invasion following siRNA transfection in OVCAR3 cells.

FIG. 9A is a bar graph depicting Caspase 3 fluorometric activity in SKOV3ip1CIS cells 72 hours after siRNA transfection. FIG. 9B is depicting Western blot analysis of apoptotic-related proteins. FIG. 9C is a bar graph depicting the quantified band intensities of cell-death proteins between the transfected cells with non-coding and CASC10 SIRNAs. FIG. 9D is a bar graph depicting the quantified band intensities of BCL-2 protein levels between the non-coding and CASC10 siRNA transfected cells. FIG. 9E is a bar graph depicting the populations of cells at different stages of cell cycle arrest. FIG. 9F is a bar graph depicting the percentage of cells at each stage of cell cycle arrest by treatment group. FIG. 9G is a Western blot analysis of cell-cycle related proteins 48 hours after SIRNA infection. FIG. 9H is a bar graph depicting the quantified band intensities of cell-cycle related proteins between treatment groups. FIG. 9I is a bar-graph depicting the quantified band intensities of Cyclin E1 between the non-coding and CASC-10 siRNA transfected cells.

FIG. 10A depicts the subcellular subcompartment analysis of CASC10 RNA levels in K562 cells. FIG. 10B is an Ingenuity Pathway Analysis (IPA) showing 25 top canonical pathway interactions following siRNA-mediated knockdown of CASC10 in OVCAR3CIS cells.

FIG. 11 depicts a flow chart of overall workflow resulting in differential gene expression analysis.

FIG. 12 is western blot images. FIG. 12A shows A) Cleaved Caspase-3 and Cleaved Caspase-9 protein expression. FIG. 12B shows PARP and Cleaved-PARP. FIG. 12C shows full Caspase-3. FIG. 12D shows BCL-2. FIGS. 12A-12D corresponds to FIG. 4B and FIG. 9B. FIG. 12E shows CDK4 protein expression. FIG. 12F shows p27 proetin expression. FIG. 12G shows Cyclin D3 protein expression. FIG. 12H shows cyclin E1 protein expression. E-H corresponds to FIG. 4G and FIG. 9G.

### DETAILED DESCRIPTION

The disclosure relates to the silencing of genes associated with ovarian cancer. In a preferred embodiment the silenced gene is CASC10

Reference will now be made in detail to exemplary embodiments of the claimed invention. While the claimed invention will be described in conjunction with the exemplary embodiments, it will be understood that it is not intended to limit the claimed invention to those embodiments. To the contrary, it is intended to cover alternatives, modifications, and equivalents, as may be included within the spirit and scope of the claimed invention, as defined by the appended claims.

Those of ordinary skill in the art may make modifications and variations to the embodiments described herein without departing from the spirit or scope of the claimed invention. In addition, although certain methods and materials are described herein, other methods and materials that are similar or equivalent to those described herein can also be used to practice the claimed invention.

In addition, any of the compositions or methods provided, disclosed, or described herein can be combined with one or more of any of the other compositions and methods provided, disclosed, or described herein.

### Definitions

Unless defined otherwise, all technical and scientific terms used herein have the meaning commonly understood by a person skilled in the art to which the claimed invention belongs. The terminology used herein is for describing particular embodiments only and is not intended to be limiting of the claimed invention. All technical and scientific terms used herein have the same meaning.

The following references provide those of skill in the art with a general understanding of many of the terms used herein (unless defined otherwise herein): Singleton et al., Dictionary of Microbiology and Molecular Biology, 3rd ed. (Wiley, 2006); Walker, The Cambridge Dictionary of Science and Technology (Cambridge University Press, 1990); Rieger et al., Glossary of Genetics: Classical and Molecular, 5th ed. (Springer Verlag, 1991); and Hale et al., Harper Collins Dictionary of Biology (HarperCollins Publishers, 1991). Generally, the procedures or methods described herein, and the like are common methods used in the art. Such standard techniques can be found in reference manuals such as, for example, Green et al., Molecular Cloning: A Laboratory Manual, 4th ed. (Cold Spring Harbor Laboratory Press, 2012), and Ausubel, Current Protocols in Molecular Biology (John Wiley & Sons Inc., 2004).

The following terms may have meanings ascribed to them below, unless specified otherwise. However, it should be understood that other meanings known or understood by those having ordinary skill in the art are also possible, and within the scope of the claimed invention. All publications, patent applications, patents, and other references mentioned or discussed herein are expressly incorporated by reference in their entireties. In the case of conflict, the present specification, including definitions, will control. In addition, the materials, methods, and examples are illustrative only and not intended to be limiting.

As used herein, the singular forms “a,” “and,” and “the” include plural references, unless the context clearly dictates otherwise.

As used herein, the term “or” means, and is used interchangeably with, the term “and/or,” unless context clearly indicates otherwise.

As used herein, the term “including” means, and is used interchangeably with, the phrase “including but not limited to.”

As used herein, the term “such as” means, and is used interchangeably with, the phrase “such as, for example” or “such as but not limited.”

Unless specifically stated or obvious from context, as used herein, the term “about” is understood as within a range of normal tolerance in the art, for example, within two standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “nucleic acid molecule” and “polynucleotide” refer to a polymer or large biomolecule comprised of nucleotides. The term “nucleic acid” includes deoxyribonucleic acid (DNA), ribonucleic acid (RNA), and analogs thereof. Non-limiting examples of nucleic acid molecules include DNA (e.g., genomic DNA, cDNA), RNA molecules (e.g., mRNA, rRNA, CRNA, IRNA), and chimeras thereof. A nucleic acid molecule can be obtained by cloning techniques or synthesized, using techniques that are known to those of skill in the art. DNA can be double-stranded or single-stranded (coding strand or non-coding strand, i.e., antisense). A nucleic acid backbone may comprise a variety of linkages known in the art, including one or more of sugar-phosphodiester linkages, peptide-nucleic acid bonds (referred to as “peptide nucleic acids” (PNA)), phosphorothioate linkages, methylphosphonate linkages, or combinations thereof. Sugar moieties of the nucleic acid may be ribose or deoxyribose, or similar compounds having known substitutions, for example, 2′ methoxy substitutions (containing a 2′-O-methylribofuranosyl moiety) and/or 2′ halide substitutions. Nitrogenous bases may be conventional bases (adenine (A), guanine (G), thymine (T), cytosine (C), and uracil (U)), known analogs thereof (e.g., inosine), known derivatives of purine or pyrimidine bases.

As used herein, the term “probe” refers to a nucleic acid oligonucleotide that hybridizes specifically to a target sequence in a nucleic acid or its complement, under conditions that promote hybridization, thereby allowing detection of the target sequence or its amplified nucleic acid. Detection may either be direct (i.e., resulting from a probe hybridizing directly to the target or amplified sequence) or indirect (i.e., resulting from a probe hybridizing to an intermediate molecular structure that links the probe to the target or amplified sequence). A probe's “target” generally refers to a sequence within an amplified nucleic acid sequence (i.e., a subset of the amplified sequence) that hybridizes specifically to at least a portion of the probe sequence by standard hydrogen bonding or “base pairing.” Sequences that are “sufficiently complementary” allow stable hybridization of a probe sequence to a target sequence, even if the two sequences are not completely complementary. A probe may be labeled or unlabeled. A probe can be produced by molecular cloning of a specific DNA sequence, or it can be synthesized. Probes for use in the methods disclosed herein can be readily designed and used by those of skill in the art.

As used herein, the term “primer” refers to a nucleic acid oligonucleotide that hybridizes specifically to a target sequence in a nucleic acid or its complement, and which is capable of priming the synthesis of a nascent nucleic acid in a template-dependent process. Primers may be provided in double-stranded or single-stranded form. Primers for use in the methods disclosed herein can be readily designed and used by those of skill in the art.

As used herein the term “siRNA” refers to small interfering RNA or “silencing RNA”. SIRNA is a class of double-stranded RNA, which are non-coding molecules. An siRNA is typically between about 20 to about 24 nucleic acid base pairs in length. In a preferred embodiment the siRNA is about 21 nucleic acid base pairs in length. An siRNA molecule degrades mRNA after transcription by interfering with expression of genes having a complementary nucleotide sequence. The result is a prevention of translation. siRNA have a phosphorylated 5′ end and hydroxylated 3′ ends and can be produced from long double stranded RNA and small hairpin RNA using an enzyme. In a preferred embodiment the enzyme is a Dicer enzyme.

As used herein the term “cancer” refers to a group of diseases that share the common characteristic of abnormal cell growth. Cancers can remain in a given location in a body or can spread throughout the body. There are more than about 200 types of cancer and are classified by where they arise in the body or type of cell from which they originate. These include carcinomas, sarcomas, leukemias, lymphoma and myelomas, blastomas, and brain and spinal cord cancers. Cancers can be benign or malignant. In a preferred embodiment the cancer is ovarian cancer. In a further preferred embodiment, the ovarian cancer is high-grade serous ovarian carcinoma (HGSOC). In a second preferred embodiment the cancer is breast cancer or inflammatory breast cancer. The cancer can also be a brain or nervous system cancer; an endocrine system cancer; a gastrointestinal cancer; a genitourinary and gynecologic cancer; a head and neck cancer: a hematopoietic cancer; a skin cancer: or a thoracic and respiratory cancer.

As used herein “OVCAR3” refers to a high-grade serous ovarian adenocarcinoma cell line. The cell line is sensitive to a variety of chemotherapeutic drugs. The cell line expresses the wilms tumor 1 protein, a marker of advanced ovarian carcinoma. The cells are known to be migratory with invasion ability.

As used herein “Cisplatin-resistant cells (OVCAR3CIS)” refer to the OVCAR cancer cell line that is resistance to the effects of cisplatin, a common chemotherapeutic drug. One of skill in the art will understand that cisplatin is used to treat a wide range of cancers including ovarian, testicular, cervical, bladder, lungs, and head and neck cancers. However, patients often develop a cisplatin resistance thereby impeding cancer treatment. Cisplatin damages cellular DNA leading to cell death.

As used herein “high-grade serous ovarian carcinoma (HGSOC)” or “high-grade serous ovarian cancer” refer to the most common and deadly type of ovarian cancer. The cancer is an epithelial ovarian cancer and cells from the HGSOC can be cultured and used in a wide variety of studies, such as those disclosed herein.

As used herein “OV-90CIS (OV-90 Cisplatin)” is a OV-90 cell line subtype that is cisplatin-resistant.

As used herein “SKOV3ip” is a metastatic human ovarian cancer cell line that lacks or has reduced levels of MKK4. SKOV3 cancers have epithelial-like morphology and are resistant to a subset of cytotoxic drugs as well as tumor necrosis factor.

### Kits

The disclosure also provides for kits comprising at least one siRNA of the invention. Kits containing an siRNA disclosed herein is useful in blocking gene expression of a particular gene as a treatment or therapy. The kit can also be used in a diagnostic assay.

Kits of the invention can the siRNA of interest, necessary buffers, plates. and pre-filled or empty syringes or other delivery vehicle. In one embodiment the invention encompasses kits for delivering a single-dose. In an alternative embodiment the kit can have a first container with a lipolyzed siRNA product and a second container having an aqueous formulation.

### EXAMPLES

The claimed invention is further illustrated by the following Examples, which should not be construed as limiting. Those of skill in the art will recognize that the claimed invention may be practiced with variations of the disclosed structures, materials, compositions, and methods, and such variations are regarded as within the scope of the claimed invention.

### Methods

### Cell Culture

High-grade serous ovarian carcinoma (HGSOC) cells OVCAR3 (NIH: OVCAR-3) and OV-90 were purchased from ATCC (Chicago, IL). Human epithelial ovarian cancer cells SKOV3ip1 were donated. Cisplatin-resistant cells OVCAR3CIS, OV-90CIS, and SKOV3ip1CIS were generated by exposing their sensitive counterpart to increasing doses of cisplatin. OVCAR3 and OVCAR3CIS were maintained in RPMI-1640 (HyClone) supplemented with 0.01 mg/mL insulin (Sigma-Aldrich), SKOV3ip1, and SKOV3ip1CIS cells were maintained in RPMI-1640 (HyClone), and OV-90 and OV90CIS were maintained on a 1:1 mixture of MCDB 105, and Medium 199 (Sigma-Aldrich). Culture media was supplemented with 10% Fetal Bovine Serum and 1% antibiotics at 37° C. in 5% CO2 and 95% O2air. All experiments were performed at 70-80% cell confluence.

### RNA-Seq and Data Analysis in HGSOC Cells

Total RNA was isolated from OVCAR3, OVCAR3CIS, OV-90, and OV90CIS cells using a mirVana™ miRNA Isolation Kit (ThermoFisher Scientific) per the manufacturer's instructions. RNA concentration and quality were verified on all samples using a NanoDrop spectrophotometer. RNA was enriched, and the library prepared using GENEWIZ® Strand-specific RNA sequencing with rRNA depletion (GENEWIZ, Inc. South Plainfield, NJ). The library was quantified with KAPA SYBR® FAST qPCR and sequenced using an Illumina HiSeq (PE 2×150 bp). Unique gene counts were calculated, and initial gene expression analysis performed using DESeq2.

### Western Blot Analysis

Cell pellets were lysed with complete lysis buffer and total protein concentration quantified using. Protein samples were separated by SDS-PAGE, blotted onto nitrocellulose membranes, blocked in either 5% non-fat dry milk (Bio-Rad) or 5% BSA (HyClone), and probed with the appropriate dilution of corresponding primary antibody. Membranes were rinsed and incubated with corresponding HRP-conjugated secondary antibody, followed by enhanced chemiluminescence and autoradiography.

### Small-Interfering RNA (siRNA) and In-Vitro Transfection

SiRNA ON-TARGET plus SMARTpool (a mixture of 4 siRNA as a single tube) and a negative control siRNA (NC-siRNA) (Sigma-Aldrich) were used for transfection studies. CASC10, was targeted by using two siRNAs targeting different regions of the CASC10 RNA (e.g., CASC10-siRNA (1) and CASC10-siRNA (2)). OVCAR3CIS or SKOV3ip1CIS cells were seeded into 12-well plates at 3.0×10⁴ cells/mL. The next day, siRNAs were mixed with HiPerfect transfection reagent (Qiagen, Valencia, CA) at a 1:2 ratio (SIRNA: HiPerfect) in serum and antibiotic-free Opti-MEM medium (Gibco) and added to the cells. After twenty-four hours the media was replaced by regular culture media, and cells were cultured and used for further experiments. To assess siRNA transfection efficiency, cells were collected 24 hours after siRNA transfection.

### Liposome Preparation

For in vivo delivery, siRNAs were mixed with DOPC (1:10 w/w), DSPE-PEG-2000 (5% mol/mol of DOPC) and cholesterol (25% w/w of DOPC) in the presence of excess ter-butanol. The mixture was frozen at-80° C. and lyophilized. Before in vivo administration, the lyophilized powder was hydrated with Ca2+ and Mg²⁺-free PBS at a concentration of 25 mg/mL to achieve the desired dose of 5 ug of siRNA in 200 pl/injection.

In some embodiments the siRNA to DOPC in the liposome preparation is about a 1:2 to about a1: 20 ratio. In other embodiments the siRNA to DOPC ratio in liposome preparation is about 1:3: about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, or about 1:20. In some embodiments the DSPE-PEG-2000concentration is about 1% to about 10% mol/mol of DOPC. In some embodiments the concentration is about 2%, about 3%, about 4%, about 5%, about 6%, about 7%, about 8%, about 9%, or about 10%. In some embodiments the cholesterol is present in the preparation in a concentration of about 10% to about 40% w/w of DOPC. In some embodiments the concentration is about 10%, about 15%, about 20%, about 25%, about 30%, about 35%, or about 40%.

### Cell Growth and Cell Viability

OVCAR3CIS cells (3.0×10⁴ cells/mL) or SKOV3ip1CIS (3.0×10⁴ cells/mL) were seeded into 12-well plates. Twenty-four hours later, cells were transfected with 100 nM (final concentration) of each siRNA. The next day, 400 cells (OVCAR3CIS) or 1,000 cells (SKOV3ip1CIS) were seeded into 6-well plates and incubated for seven days, fixed and stained with 0.5% crystal violet in methanol. Colonies of at least 50 cells were counted using a light microscope (CKX41: Olympus, Center Valley, PA, USA) with a total magnification of 40x. For cell viability assays, OVCAR3 and OVCAR3CIS (3.0×10⁴ cells/mL) or SKOV3ip1and SKOV3ip1CIS (3.5×10⁴ cells/mL) were seeded into 96-well plates, and 24 hours later, siRNA transfection was performed as described above. The next day, the transfection mix was replaced with cisplatin (CIS) (2.5 uM final concentration dissolved in regular cell culture media) and after forty-eight hours, the medium was removed, and Alamar blue dye (Invitrogen, Thermo Fisher Scientific, Eugene, OR, USA) was added following the manufacturer's instructions. Optical density (OD) was measured, and percentages of cell viability were calculated after blank OD subtraction. Untreated cells were assessed as 100% cell viability.

### Cell Invasion

Cell invasion was measured using the Matrigel transwell method. OVCAR3CIS (3.0×10⁴ cells/mL) or SKOVip1CIS (3.5×10⁴ cells/mL) were seeded into 10 cm Petri dishes and transfected with siRNAs. Twenty-four hours later, serum-free matrigel (BD Biosciences CA, USA) was added onto the upper chambers of 24-well plates and incubated at 37° C. for polymerization. Transfected cells were collected and resuspended in serum-free and re-seeded onto the Matrigel-coated chambers. Medium containing 10% FBS was added to the lower area of the wells, and the plates incubated for 48 hours at 37° C. The medium was removed, and cells that invaded through the matrigel fixed and stained using ProtocolHema 3 Stain Set (Fisher Scientific MI, USA). Invaded cells were microscopically counted using a digital camera to capture images at a 20× resolution. The percentage of cell invasion was calculated using the NC-SIRNA condition as 100% cell invasion.

### Caspase-3 Activity

Caspase-3 activity was quantified using the Caspase-3/CPP32 Fluorometric Assay Kit (BioVision CA, USA) as per the manufacturer's instructions. OVCAR3CIS (3.0×10⁴ cells/mL) or SKOV3ip1CIS (3.5×10⁴ cells/mL) were seeded into 10 cm Petri dishes and transfected with NC-siRNA or CASC10-siRNA (2). Twenty-four hours later, the media was replaced by regular media, and seventy-two hours after transfection, cells were collected, pellets lysed, and total protein concentration determined. Equal amounts of protein were mixed with 2×Reaction Buffer and 1 mM DEVD-AFC substrate in a 96-well plate and incubated at 37° C. for 2.5 hours. Fluorescence intensity at 400 nm excitation and 505 nm emission was measured.

### KM Plotter Database Interrogation

Kaplan-Meier survival analysis was performed using available patient datasets from gene chip and RNA-seq in the internet searchable database, Kaplan-Meier (KM) plotter [8]. For each gene, ovarian cancer patients were divided into high and low expression groups by the median value of their RNA expression. A set of different filters was applied, including ovarian cancer patients, ovarian cancer patients treated with platinum, and serous ovarian cancer patients treated with platinum. Kaplan-Meier survival plots for overall survival (OS) and progression-free survival (PFS) were obtained with their respective hazard ratios (HR), confidence intervals (CI), and p-values (log-rank). For these studies, p-values <0.05 were considered statistically significant.

### SYBR-Green Based qRT-PCR

A custom-made 384-well plate containing pre-designed forward and reverse primers was purchased from Bio-Rad (CA, USA). Total RNA was isolated from OVCAR3 and OVCAR3CIS cells using the GenElute Mammalian Total RNA Mini Kit (Millipore-Sigma, MO, USA) following the manufacturer's instructions. RNA was reverse transcribed using the iScript Reverse Transcription Supermix for RT-qPCR from Bio-Rad. SYBR Green-based qPCR was performed using the SsoAdvanced™ Universal SYBR® Green Supermix (Bio-Rad) and a CFX384 Touch Real-Time PCR detection system. Fold-changes and cycle threshold (Ct) values were calculated by the instrument's internal software relative to OVCAR3 cells and normalized to B-actin along with controls for gDNA, PCR reaction, RT reaction, and RNA quality.

### Flow Cytometry

To assess cell cycle progression, OVCAR3CIS (3.0×10⁴ cells/mL) or SKOV3ip1CIS (3.5×10⁴ cells/mL) were seeded into 10 cm Petri dishes and transfected with NC-siRNA or CASC10-siRNA (2). Forty-eight hours later, attached cells were collected, washed in ice-cold PBS, fixed with 70% cold ethanol, and stored at 4° C. Cells were then washed with ice-cold PBS, resuspended in propidium iodide (PI)/RNase Staining Buffer, incubated in the dark for 15 minutes at room temperature, and then analyzed by flow cytometry in BD C6 Accuri (CA, USA). Accuri's software was used to determine the percentage of cells in each cell cycle phase.

### Tumor Implantation and Drug Treatment

Female athymic nude mice (NCr-nu, 6 weeks old) were used to assess the therapeutic efficacy of liposomal CASC10-siRNA (CASC10-siRNA (2)) alone or in combination with Cisplatin (CIS) in vivo. Mice were intraperitoneally (i.p) injected with OVCAR3CIS (1.5×10⁸ cells/0.2 mL HBSS). Seven days later, mice were randomly divided into the following treatment groups (N=10 per group): (a) NC-siRNA, (b) CIS alone, (c) CASC10-SIRNA, (d) NC-siRNA plus CIS, and (e) CASC10-siRNA (2) plus CIS. Liposomal SiRNAs (10 μg siRNA/injection) and CIS (160 μg/injection) were injected (i.p) twice a week for four weeks.

At the end of the treatment, mice were euthanized, tumors were collected, and the number of tumor nodules and tumor weight were recorded. Animal handling and research protocols were approved by the Institutional Animal Care and Use Committee (IACUC) of the University of Puerto Rico, Medical Sciences Campus.

### RNA-seq Analysis of CASC10-siRNA Transfected Cells

The RNA sequencing library was prepared by first extracting, total RNA using the GenElute Mammalian Total RNA Miniprep Kit (Sigma). RNA sample integrity was evaluated and RNAs with a RIN >7 were used.

RNA fragmentation was carried out for 10 minutes, followed by first-and second-strand cDNA synthesis and adenylation using the TruSeq Stranded Total RNA with Illumina Ribo-Zero Plus rRNA Depletion (Illumina, San Diego, CA). The PCR products were run on a flow cell running a 50 base paired-end (2X50) recipe. The differential expression analysis was carried out using the DESeq2 (version 1.28.1) package (R version 4.0.1). As the count data was obtained in two batches, a batch correction term was introduced in the DESeq2 model using ComBat Seq to have better statistical power and control of false positives. The Ensembl IDs were converted to gene symbols and names using the org.Hs.eg.db package (version 3.11.4). Significance was set at an FDR-adjusted p-value <0.01 and log2 fold change >1.

### Statistical Analysis

All experiments were performed at least in triplicates. Graphing and statistical analysis were performed using the GraphPad Prism (CA, USA) software 9.3.1. Data were analyzed using Student's t-test for comparing two groups and ANOVA tests for multiple comparisons, with p <0.05 considered statistically significant.

### Results

Identification of differentially expressed transcripts in cisplatin-sensitive versus cisplatin-resistant HGSOC cells

The IC50 of the ovarian cancer cell lines has been previously reported and is summarized in Table 1.

TABLE 1
Concentrations of cisplatin inhibiting 50% cell viability.
Incubation with cisplatin: 72-hr followed by Alamar Blue assay

| Cell line | IC50 cisplatin (μM) |
|---|---|
| A2780 | 0.5-0.8 |
| A2780CP20 | 20-30 |
| A2780CIS | 4.3 |
| OV90 | 3.5 |
| OV90CIS | 7.8 |
| OVCAR3 | 2.8 |
| OVCAR3CIS | 7.5 |
| SKOV3IP1 | 3.9 |
| SKOV3IP1CIS | 7.3 |

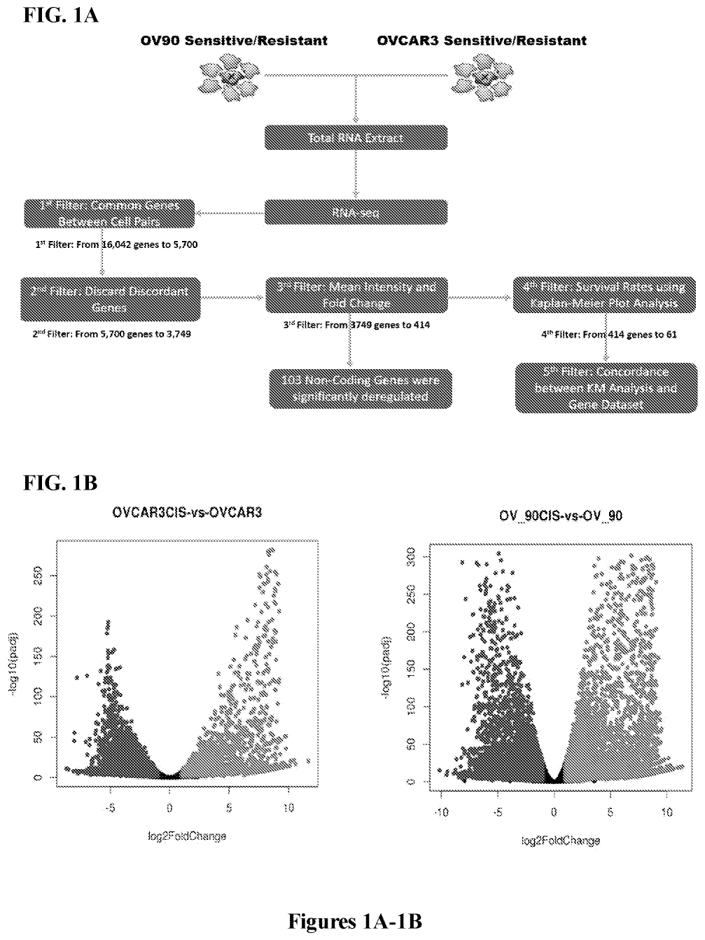

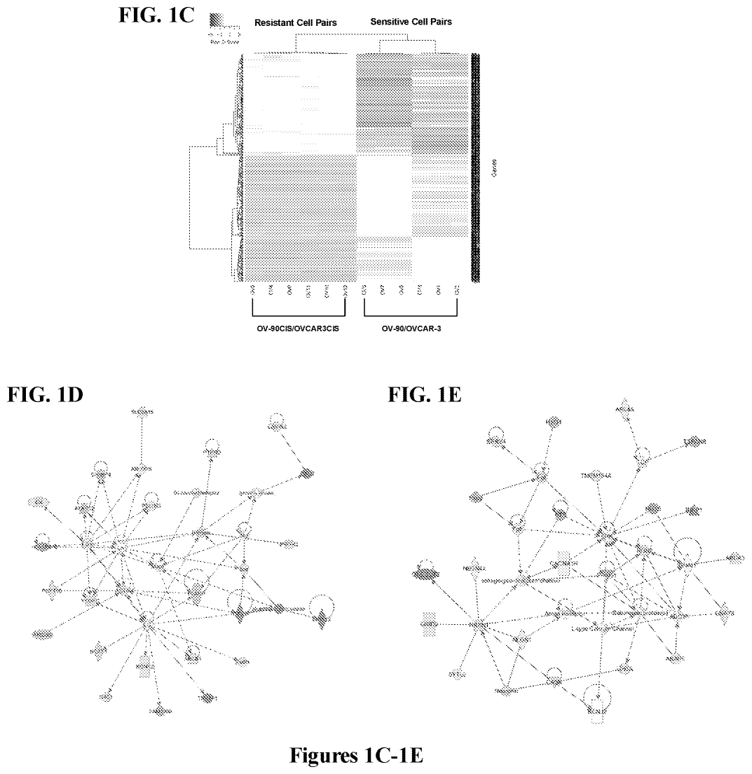

To identify differentially abundant RNA transcripts in HGSOC cells, RNAseq was performed in OV-90 and OVCAR3 and their cisplatin-resistant counterparts OV-90CIS and OVCAR3CIS cells. A diagram of how the RNAseq data was filtered is shown in FIG. 1A. A total amount of 10,714 significant (p<0.05) differentially abundant transcripts (DEGs) in OV-90/OV-90CIS and 5,328 in OVCAR-3/OVCAR3CIS were initially identified (see GEO, in progress, which contains the complete list of identified transcripts). The volcano plot shown in FIG. 1B indicates that several genes were differentially abundant (right side of each plot): increased and decreased (left side of each plot) in the cisplatin-resistant as compared with the cisplatin-sensitive cells (FIG. 1B). In a first data filtering, genes identified in only one pair of cell lines were eliminated while genes common to both cell lines remained. This reduced the list to 5,700 RNA transcripts commonly expressed in both cell line pairs. A second filter was introduced to eliminate the transcripts that showed opposite expression tendency in both cell lines (i.e., upregulated in one pair of cell lines and downregulated in the other pair of cells), which reduced the list to 3,749 deregulated transcripts. Next, a third filter was performed based on the distributions of the base mean intensity of the transcript and fold change. Each transcript was ranked (Rank range: 2-8) by adding the numbers corresponding to the quartile of the distribution where the value of both the base mean intensity and fold change lie within the distribution (1-4 for each distribution). For this analysis, we selected all transcripts with a rank ≥7, which reduced the number of transcripts from 3,749 to 414 (237 upregulated and 177 downregulated in both cell pairs); see Table 2 with the list of the 414 transcripts.

TABLE 2
List of the 414 transcripts most differentially abundant in cisplatin
sensitive vs. cisplatin resistant HGSOC cells.

| | OV90log2FC | OVCAR3log2FC |
|---|---|---|
| Gene Name | (Resistant/Sensitive) | (Resistant/Sensitive) |
| BCAT1 | 2.849 | 4.562 |
| SH3BP4 | 3.63 | 3.794 |
| NABP1 | −2.426 | −5.027 |
| FGF2 | −5.443 | −2.098 |
| STARD4 | −4.152 | −3.39 |
| RGS2 | 2.609 | 4.942 |
| ID4 | −6.583 | −0.976 |
| PCYT1B | −6.865 | −0.760 |
| THBS1 | 3.589 | 4.061 |
| LMO7 | 5.368 | 2.285 |
| TENM3 | −2.439 | −5.270 |
| AKAP12 | 3.951 | 3.783 |
| SYTL2 | −3.189 | −4.547 |
| CYP1B1 | 3.069 | 4.801 |
| F2RL1 | 3.652 | 4.226 |
| ATP11B | −3.213 | −4.713 |
| OGFRL1 | −3.043 | −4.917 |
| SLC39A8 | −3.54 | −4.474 |
| TNFAIP2 | 6.296 | 1.946 |
| CRABP2 | −6.656 | −1.622 |
| PAX9 | −5.501 | −2.779 |
| NLGN1 | −3.428 | −5.05 |
| ANKRD1 | −2.663 | −5.829 |
| PDE1A | 0.964 | 7.528 |
| QPRT | 5.511 | 3.016 |
| TMEM30B | −6.499 | −2.105 |
| GDF15 | 4.966 | 3.659 |
| SP3P | −3.396 | −5.275 |
| ITM2B | 4.733 | 3.947 |
| CA2 | 6.825 | 1.868 |
| PCDH17 | −4.544 | −4.152 |
| BRINP3 | −6.77 | −1.955 |
| AKAP6 | −4.71 | −4.035 |
| ANTXR2 | 7.893 | 0.904 |
| MMP16 | −6.169 | −2.687 |
| NRP1 | 7.714 | 1.173 |
| A2M | 7.737 | 1.177 |
| EFEMP1 | 8.179 | 0.765 |
| ABCC2 | 7.886 | 1.073 |
| ZDBF2 | −4.461 | −4.553 |
| PRSS23 | 6.641 | 2.391 |
| MUC 15 | −6.231 | −2.856 |
| ARL4A | −3.648 | −5.454 |
| SLIT2 | −7.061 | −2.093 |
| MID1 | −3.178 | −6.01 |
| FAM198B | 4.885 | 4.342 |
| CCL26 | 8.556 | 0.711 |
| CDH6 | 8.573 | 0.742 |
| FAS | −5.727 | −3.592 |
| PROM1 | 7.886 | 1.436 |
| LIPG | 3.67 | 5.678 |
| NAALAD2 | 2.989 | 6.403 |
| LIN28A | 1.378 | 8.027 |
| MMRN1 | 0.752 | 8.664 |
| HIST1H2BH | 0.755 | 8.683 |
| RBM47 | 6.572 | 2.887 |
| SLC7A7 | 7.533 | 1.93 |
| FGG | 8.522 | 1.004 |
| ZFP42 | 0.876 | 8.785 |
| GLDC | −6.067 | −3.595 |
| NRK | 0.705 | 9.013 |
| JAG1 | 8.212 | 1.523 |
| ISL1 | −4.549 | −5.2 |
| LAMC2 | 8.494 | 1.367 |
| NID1 | 5.37 | 4.504 |
| RP3-332B22.1 | −6.373 | −3.508 |
| FRY | −5.233 | −4.715 |
| ZPLD1 | 1.86 | 8.16 |
| LHX8 | 1.083 | 8.985 |
| MSX1 | 1.8 | 8.323 |
| CD24 | 8.619 | 1.508 |
| ADGRL2 | −4.868 | −5.26 |
| POU1F1 | −6.368 | −3.783 |
| LAMA2 | −4.911 | −5.244 |
| RP11-115D19.1 | 1.062 | 9.22 |
| MBNL2 | 6.276 | 4.028 |
| ADAM28 | 8.719 | 1.589 |
| CRISPLD1 | −6.566 | −3.755 |
| TM4SF1 | 8.555 | 1.792 |
| C3orf58 | −5.97 | −4.393 |
| AARD | −6.38 | −3.992 |
| C1S | 6.658 | 3.802 |
| RHOBTB1 | 3.937 | 6.563 |
| SULF1 | −6.791 | −3.781 |
| HIST1H3G | 1.632 | 8.958 |
| ACTA2 | −7.269 | −3.338 |
| SEMA3D | −6.206 | −4.422 |
| CLDN16 | −5.611 | −5.292 |
| SPP1 | 7.785 | 3.136 |
| GRID2 | −5.303 | −5.68 |
| ARID5B | 3.283 | 7.713 |
| ANK3 | 3.591 | 7.446 |
| IQGAP2 | 6.336 | 4.8 |
| GNAT3 | −7.125 | −4.09 |
| HDAC9 | 3.747 | 7.556 |
| TOX | −6.581 | −4.901 |
| MMP3 | −6.707 | −4.834 |
| VGLL3 | −6.179 | −5.417 |
| RGS1 | 2.701 | 8.926 |
| GPRIN3 | 2.905 | 8.771 |
| MLIP | −6.497 | −5.213 |
| SIM1 | −6.263 | −5.48 |
| SDPR | −6.553 | −5.299 |
| NRG1 | −6.513 | −5.384 |
| BICC1 | 4.73 | 7.199 |
| SYNPO2 | −6.801 | −5.135 |
| SEMA3E | −7.43 | −4.543 |
| SLC8A1 | 3.705 | 8.342 |
| TENM2 | −7.826 | −4.225 |
| HIST1H3C | 8.647 | 3.475 |
| TMEM2 | 5.24 | 7.101 |
| AMIGO2 | 3.685 | 8.893 |
| NTS | 6.103 | 6.532 |
| WDR72 | 8.587 | 4.104 |
| NUPR1 | 6.008 | 6.844 |
| CAV2 | 8.594 | 4.388 |
| PDGFRA | 8.246 | 4.799 |
| TGFB2 | 5.758 | 7.377 |
| HIST1H2BE | 5.774 | 7.457 |
| SATB1 | 4.759 | 8.473 |
| CARD16 | 4.871 | 8.633 |
| FUCA2 | 8.236 | 5.388 |
| MEST | 5.238 | 8.612 |
| HSPA1A | 7.348 | 6.879 |
| HIST1H2BI | 6.462 | 8.298 |
| UNC13C | 7.1 | 8.031 |
| HIST1H2AL | 7.753 | 7.459 |
| ZNF532 | 7.147 | 8.119 |
| TES | 7.492 | 7.874 |
| HIST1H31 | 7.912 | 7.533 |
| HIST1H4D | 7.189 | 8.266 |
| HIST1H1B | 7.685 | 8.058 |
| DKK1 | 7.451 | 8.426 |
| FTH1 | 2.155 | 1.679 |
| PNMAL1 | −3.203 | −0.632 |
| SACS | −2.808 | −1.027 |
| MAP1A | 2.177 | 1.674 |
| ARRDC3 | −1.682 | −2.252 |
| SESN3 | −0.533 | −3.407 |
| ARL6IP5 | −2.926 | −1.04 |
| KLF3 | 2.697 | 1.272 |
| TMCO3 | 2.469 | 1.514 |
| ABRACL | −2.029 | −1.963 |
| CASK | 2.897 | 1.102 |
| CRIM1 | −2.361 | −1.655 |
| GJA1 | −3.285 | −0.789 |
| SORT1 | 2.224 | 1.856 |
| ADAM15 | −3.681 | −0.428 |
| FAM234B | −2.457 | −1.682 |
| U1 | −3.686 | −0.496 |
| HMGB1P6 | −3.098 | −1.088 |
| OCLN | −0.438 | −3.757 |
| HSPA4L | −2.824 | −1.39 |
| SPRY4 | 2.373 | 1.907 |
| EMP3 | 2.442 | 1.88 |
| CMBL | 3.615 | 0.71 |
| ANKRD10 | 2.715 | 1.7 |
| SLC40A1 | 3.524 | 0.932 |
| DDAH1 | −3.514 | −0.965 |
| TNFRSF19 | −2.188 | −2.369 |
| KLHL5 | 1.834 | 2.727 |
| SLC9A7 | −0.966 | −3.632 |
| CCDC138 | −2.347 | −2.258 |
| SSX2IP | −1.997 | −2.616 |
| BCHE | −3.329 | −1.31 |
| HSPG2 | 3.957 | 0.747 |
| PLAUR | 3.63 | 1.076 |
| MEIS2 | 2.529 | 2.209 |
| PIK3CA | −2.523 | −2.357 |
| PSD3 | −2.396 | −2.506 |
| TTC39A | −3.818 | −1.105 |
| ERRFI1 | 3.773 | 1.328 |
| GCAT | −3.073 | −2.125 |
| EMP1 | −4.032 | −1.205 |
| LIF | −3.039 | −2.24 |
| SLC12A6 | −2.707 | −2.612 |
| MYO5A | −3.998 | −1.396 |
| IRS1 | 4.252 | 1.218 |
| CFL2 | −3.496 | −1.976 |
| USP53 | −1.397 | −4.087 |
| MID1IP1 | 3.254 | 2.232 |
| HEG1 | 4.39 | 1.12 |
| NUP210 | −3.884 | −1.669 |
| FTL | 4.429 | 1.148 |
| INSIG2 | 2.282 | 3.31 |
| ATP11A | 4.262 | 1.333 |
| ARMCX6 | −2.951 | −2.659 |
| KONJ2 | −0.944 | −4.697 |
| KITLG | 4.409 | 1.269 |
| TBC1D32 | −3.796 | −1.935 |
| ACSL1 | −4.365 | −1.368 |
| CITED2 | −2.153 | −3.59 |
| LGR4 | 3.476 | 2.299 |
| PCDH9 | −2.982 | −2.859 |
| PCGF5 | −0.707 | −5.141 |
| APOC1 | 4.111 | 1.749 |
| KANK1 | −0.771 | −5.112 |
| ADGRG6 | −0.969 | −4.949 |
| ARHGAP28 | −4.771 | −1.238 |
| PAX6 | −1.634 | −4.429 |
| ABCA1 | 1.447 | 4.634 |
| CPED1 | 0.989 | 5.124 |
| PLAU | 4.736 | 1.385 |
| ABCA3 | −5.623 | −0.53 |
| EYA4 | −5.638 | −0.581 |
| CFH | −3.3 | −2.926 |
| TOM1L2 | −3.066 | −3.174 |
| ARAP2 | −1.707 | −4.549 |
| AMOT | 4.067 | 2.21 |
| IL1RAP | −1.151 | −5.142 |
| TSPAN8 | 5.502 | 0.827 |
| CALCRL | −5.567 | −0.867 |
| PBX1 | 4.161 | 2.288 |
| CASC10 | −6.033 | −0.497 |
| HOXB9 | −1.628 | −4.934 |
| MTUS1 | 2.154 | 4.438 |
| FLRT3 | −1.312 | −5.297 |
| SIX1 | −5.996 | −0.722 |
| OXCT1 | −5.918 | −0.829 |
| MPDZ | −5.772 | −0.996 |
| DNAJC22 | 5.946 | 0.843 |
| GNAM1 | −1.418 | −5.373 |
| HIC2 | 5.971 | 0.861 |
| RGS5 | 6.339 | 0.575 |
| PCDH7 | −1.697 | −5.227 |
| ADAMTS3 | −6.244 | −0.715 |
| NFKBIZ | 4.895 | 2.12 |
| SNRPN | −6.44 | −0.589 |
| ELOVL4 | −4.846 | −2.184 |
| VCAN | 1.392 | 5.639 |
| MET | 4.825 | 2.207 |
| CDH12 | 6.2 | 0.865 |
| SLC7A2 | 1.712 | 5.473 |
| LRP1B | −2.117 | −5.17 |
| APOE | 5.742 | 1.55 |
| F2R | 3.526 | 3.801 |
| NEFH | −6.276 | −1.054 |
| RNLS | −2.753 | −4.604 |
| PLB1 | 5.712 | 1.654 |
| XK | −2.238 | −5.142 |
| RUNX2 | 0.686 | 6.85 |
| TP63 | −4.481 | −3.09 |
| PAG1 | 4.002 | 3.577 |
| ATP8B1 | 5.935 | 1.666 |
| TOR4A | 6.637 | 0.965 |
| TDRP | −6.636 | −1.074 |
| RERG | 3.067 | 4.657 |
| HMCN1 | 5.949 | 1.803 |
| SULF2 | 5.871 | 1.882 |
| SCIN | −2.704 | −5.048 |
| C10orf10 | 5.244 | 2.526 |
| SOSTDC1 | 3.635 | 4.183 |
| MGAT3 | 4.748 | 3.083 |
| C2orf72 | 5.504 | 2.334 |
| TMCC3 | 5.595 | 2.255 |
| S1PR1 | 0.768 | 7.114 |
| PALD1 | −7.191 | −0.707 |
| MCOLN2 | −6.298 | −1.61 |
| PROCR | 2.216 | 5.769 |
| CCDC74A | −6.246 | −1.759 |
| CFP | 6.971 | 1.163 |
| PDGFC | −3.449 | −4.711 |
| RNF144B | −6.53 | −1.645 |
| VIL1 | 6.498 | 1.683 |
| NYNRIN | 4.242 | 3.95 |
| TMEM178A | −4.283 | −3.915 |
| VTN | 7.014 | 1.203 |
| TGFBR3 | 3.754 | 4.464 |
| ADGRB3 | −6.687 | −1.532 |
| ZNF415 | 4.641 | 3.591 |
| ZNF704 | 2.274 | 5.973 |
| SULT1A1 | 4.823 | 3.435 |
| CACNA1H | −3.147 | −5.151 |
| ESPN | −4.683 | −3.694 |
| NUDT11 | −6.273 | −2.126 |
| LPAR1 | −3.441 | −5.001 |
| CCDC74B | −6.883 | −1.585 |
| CDH10 | −7.619 | −0.872 |
| COL17A1 | 6.168 | 2.348 |
| NR3C1 | 7.019 | 1.502 |
| FAM184A | −3.73 | −4.797 |
| RGL1 | 4.965 | 3.578 |
| KIF1A | −7.199 | −1.433 |
| HPGD | 7.569 | 1.277 |
| SLITRK6 | −6.079 | −2.77 |
| TMEM184A | −3.868 | −4.986 |
| MMP14 | 7.021 | 1.885 |
| LRRC17 | 7.498 | 1.424 |
| ABCB1 | −4.17 | −4.788 |
| SH2D5 | −6.523 | −2.51 |
| CFTR | 7.291 | 1.814 |
| NRXN1 | −5.791 | −3.448 |
| PIK3C2G | 7.315 | 2.103 |
| PPP1R1C | 4.773 | 4.724 |
| FAT3 | 7.963 | 1.588 |
| TRPM3 | 1.672 | 7.897 |
| SLC5A5 | 7.696 | 1.898 |
| RP1-261D10.2 | −5.256 | −4.339 |
| IGFBP4 | 6.89 | 2.723 |
| SYNE4 | −5.479 | −4.176 |
| SLC16A14 | −6.305 | −3.386 |
| TFEC | 5.917 | 3.817 |
| EPAS1 | 6.717 | 3.019 |
| FBXL7 | −7.228 | −2.513 |
| NLGN4X | 0.932 | 8.871 |
| LINC01351 | −6.664 | −3.153 |
| PCLO | −6.521 | −3.312 |
| BST2 | 3.65 | 6.185 |
| GRIK1 | 2.112 | 7.739 |
| LSAMP | 8.005 | 1.852 |
| DISC1FP1 | −5.6 | −4.293 |
| NPNT | 7.795 | 2.108 |
| HIST1H4L | 8.824 | 1.158 |
| PTGIS | 7.141 | 2.896 |
| OTOA | 1.426 | 8.664 |
| KCNT2 | −5.007 | −5.086 |
| ARL10 | 3.691 | 6.456 |
| PPARGC1A | 8.514 | 1.695 |
| DNAJC6 | −4.159 | −6.053 |
| MXRA8 | 6.126 | 4.138 |
| RUNX1 | 8.628 | 1.643 |
| PDLIM3 | −4.761 | −5.558 |
| ONECUT2 | 8.279 | 2.046 |
| EPHA5 | −7.009 | −3.321 |
| HIST1H2BB | 5.909 | 4.44 |
| IGFBP3 | −7.315 | −3.037 |
| CCDC110 | −5.467 | −4.905 |
| FZD8 | 4.698 | 5.787 |
| SLITRK5 | −7.027 | −3.462 |
| VWDE | −5.336 | −5.231 |
| TRPV2 | 6.268 | 4.389 |
| TBX15 | −6.049 | −4.642 |
| CFAP47 | −5.781 | −4.912 |
| LRG1 | 7.975 | 2.723 |
| EPDR1 | −6.742 | −3.985 |
| TPH2 | −2.766 | −8.037 |
| HIST1H3J | 7.052 | 3.78 |
| TRHDE | −3.021 | −7.824 |
| PTPRD | 3.016 | 7.876 |
| MYEOV | 8.133 | 2.771 |
| ZNF788 | 1.749 | 9.172 |
| VEGFC | −6.108 | −4.837 |
| RASSF9 | 5.881 | 5.08 |
| CAV1 | 5.354 | 5.612 |
| USP44 | 7.722 | 3.286 |
| CDCP1 | 8.535 | 2.478 |
| TNS3 | 7.512 | 3.554 |
| FAM110C | −7.228 | −3.839 |
| SNTG1 | −6.821 | −4.248 |
| FMN1 | −6.023 | −5.088 |
| FAM111B | 6.931 | 4.201 |
| GS1-600G8.3 | −6.687 | −4.473 |
| PDE4B | −6.284 | −5.017 |
| SPAG6 | −7.059 | −4.258 |
| SLC6A15 | −5.882 | −5.452 |
| DCDC1 | −6.34 | −5.004 |
| ELF3 | 7.918 | 3.49 |
| GAS1 | −6.48 | −5.013 |
| NID2 | 5.152 | 6.351 |
| EOMES | 3.634 | 7.871 |
| INPP1 | 8.383 | 3.132 |
| GSG1 | −6.509 | −5.125 |
| PRRX1 | −7.367 | −4.314 |
| SV2A | 3.705 | 7.979 |
| SATB2 | −6.778 | −4.926 |
| TMEM207 | −5.756 | −5.968 |
| ZNF521 | 2.909 | 8.816 |
| LIN7A | −7.011 | −4.716 |
| MAGI2-AS3 | −7.043 | −4.79 |
| KIFC3 | 7.583 | 4.277 |
| AF121898.3 | −6.78 | −5.114 |
| ADAMTS9 | 8.532 | 3.404 |
| GALNT13 | −7.169 | −4.782 |
| ACSS3 | −6.822 | −5.131 |
| MUSK | −6.5 | −5.476 |
| SLC9A9 | −6.374 | −5.631 |
| CASP4 | 5.813 | 6.255 |
| CPA4 | 7.797 | 4.435 |
| RP11-86516.2 | −7.152 | −5.089 |
| TMEFF2 | −8.146 | −4.125 |
| PRKG1 | 5.656 | 6.652 |
| RP11-255H23.2 | 3.494 | 8.884 |
| NRG3 | 9.075 | 3.33 |
| ZNF595 | 4.361 | 8.078 |
| SLC44A5 | 7.692 | 4.886 |
| AGMO | 5.557 | 7.102 |
| PDGFD | 5.224 | 7.436 |
| SOX9 | 8.431 | 4.234 |
| ELFN1 | 4.608 | 8.097 |
| MGST2 | 8.215 | 4.519 |
| TGFB2-AS1 | 5.167 | 7.593 |
| RP11-2E11.6 | 4.689 | 8.176 |
| PTPRQ | −6.108 | −6.981 |
| OLFML3 | 4.882 | 8.284 |
| PLBD1 | 5.84 | 7.566 |
| AVPR1A | 8.135 | 5.296 |
| ZNF841 | 7.715 | 5.867 |
| ESRRG | 5.439 | 8.144 |
| ZNF681 | 5.459 | 8.4 |
| FLNC | 5.98 | 7.962 |
| HSPB1 | 6.297 | 7.656 |
| S100A11 | 7.449 | 6.726 |
| TMEM156 | 8.629 | 5.626 |
| GPX1 | 7.616 | 6.787 |
| TGFB1I1 | 7.35 | 7.098 |
| MDFIC | 8.361 | 6.556 |
| ALDH2 | 7.644 | 7.304 |
| HIST1H4A | 8.143 | 6.869 |
| ZNF320 | 6.905 | 8.371 |
| ZNF468 | 8.079 | 7.213 |
| C14orf169 | 7.129 | 8.282 |
| HIST1H3A | 8.105 | 7.314 |
| ZNF718 | 6.764 | 8.705 |

A heat map was constructed with the 414 DEGs to evaluate the transcriptional differences between resistant and sensitive cell pairs (FIG. 1C; blue represents downregulated genes and yellow represents upregulated genes). A clear difference between the upregulated and downregulated genes among the cisplatin-resistant and cisplatin-sensitive cells is observed. To visualize molecular interactions between the deregulated genes, the list with the 414 transcripts was analyzed by Ingenuity Pathway Analysis (IPA), resulting in 25 different networks (Table 3).

TABLE 3
CASC10 downstream signaling pathways generated by IPA

| Network | Diseases and Function | Molecules |
|---|---|---|
| 1 | Cell Death and Survival | TPPP, PYM1, CD151, MAGOH, |
| | Cellular Movement | NRSN1, GLYR1, TMEM64, CELF1, |
| | Developmental | PTBP1, MYC, SERINC3, Cyclin |
| | Disorders | D, SNHG3, EPHA4, CDC42, , |
| | | MERTK, CDK4, PDLIM3, CDCA7L, |
| | | TNF, CNN1, CDC42EP4, |
| | | AQP3, TRIM5, FSH, ARHGAP1, |
| | | IFRD2, WT1, SRY, SLC20A1, |
| | | EFNA1, TNFRSF6B, TMBIM6, |
| | | ETV5 |
| 2 | Cell Cycle and | PCDH7, COL4A1, SMAD6, ANXA9, |
| | Chromosomal | COL6A2, COL4A2, COL5A1, |
| | Regulation | NFIB, HMGA1, ESTROGEN |
| | Cancer and | RECEPTOR, NORAD, EGLN2, |
| | Cell Survival | ABCA1, TIMP2, KRT18, PDGFA, |
| | | TGFBR2, FGFR3, CRNDE, LTB, |
| | | SP1, RHOB, LIPA, NCOR2, |
| | | GADD45B, CR2, RB1, |
| | | NOX4, SIN3A, CDC37, |
| | | KDMSA, SIN3B, MORF4L1, |
| | | MORF4L2, MRFAP1 |

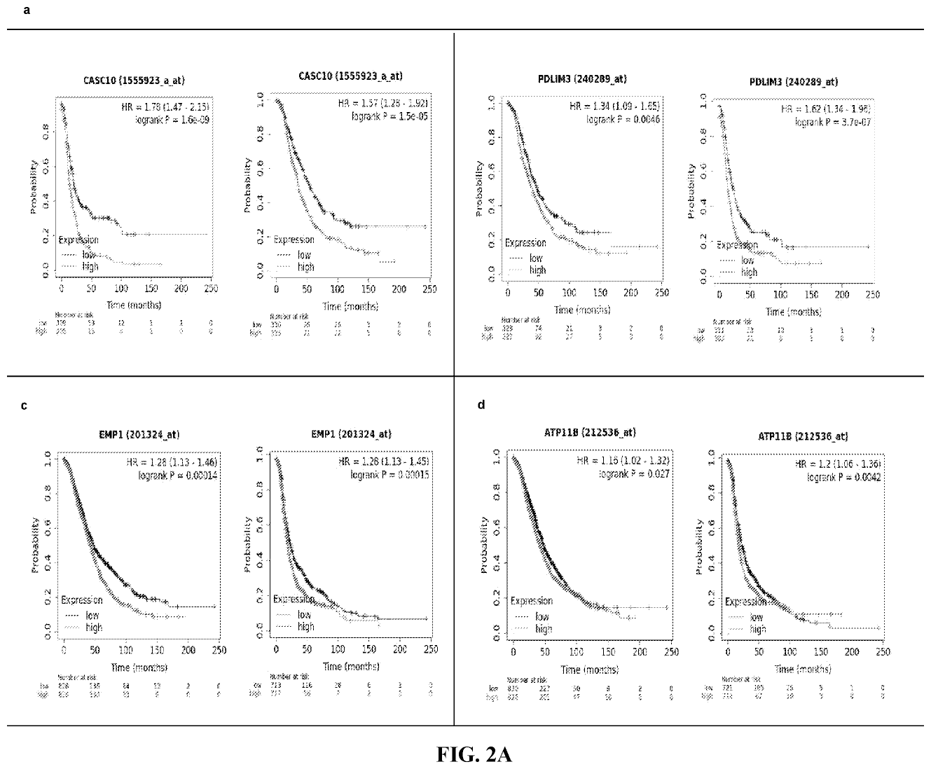

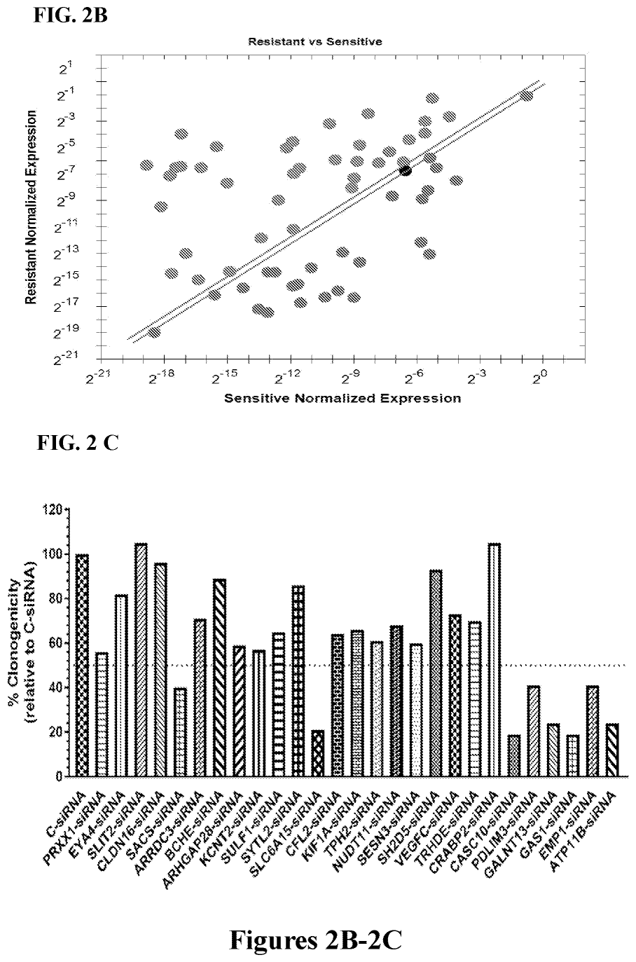

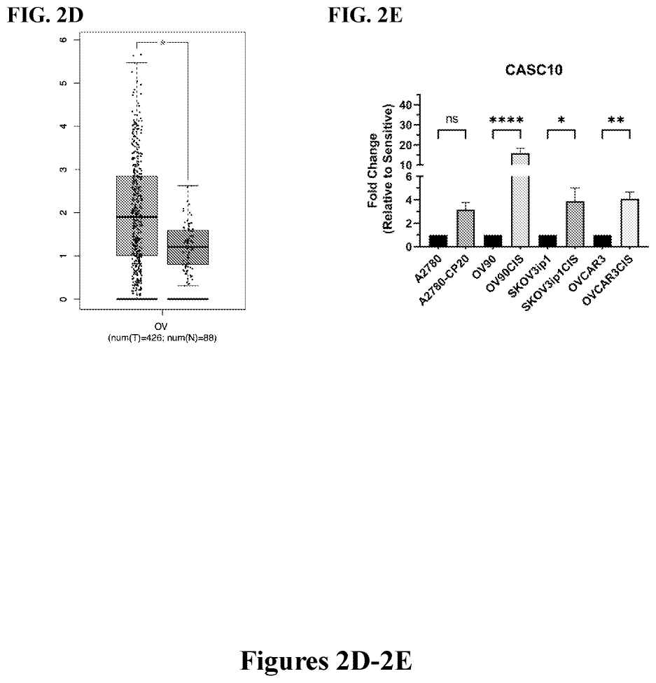

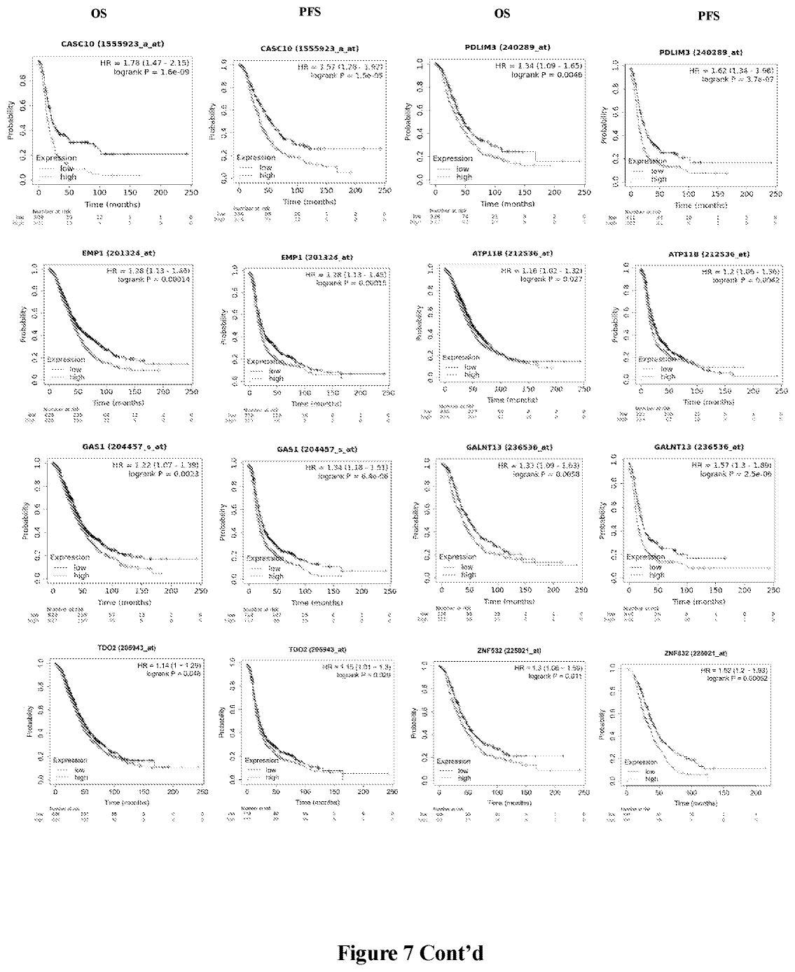

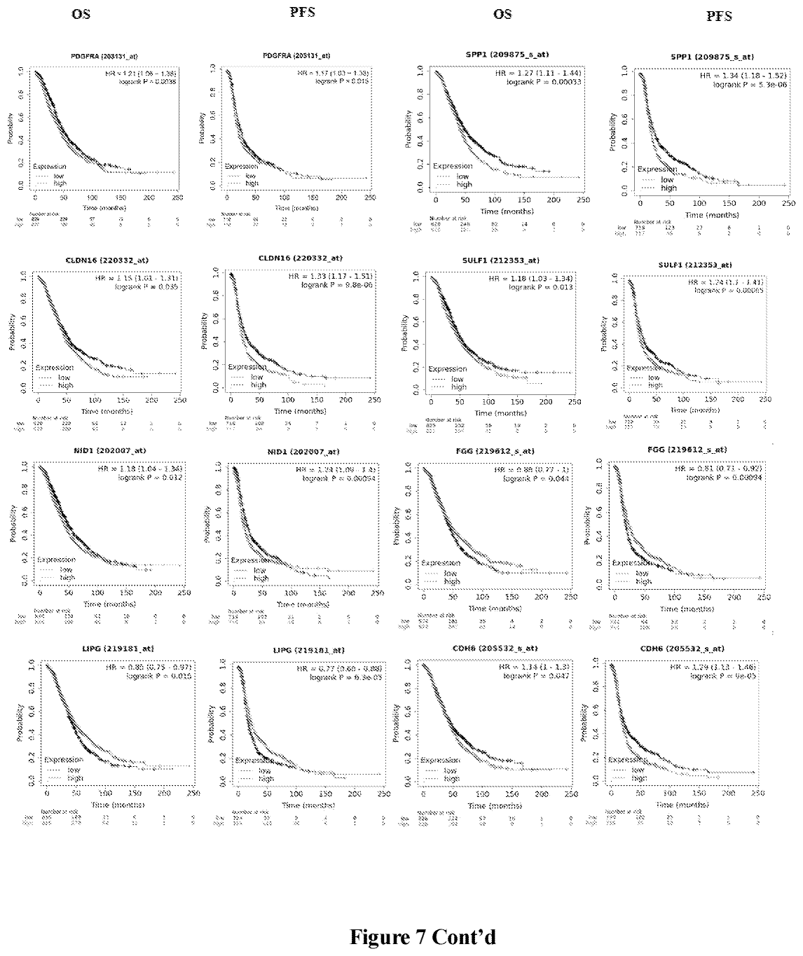

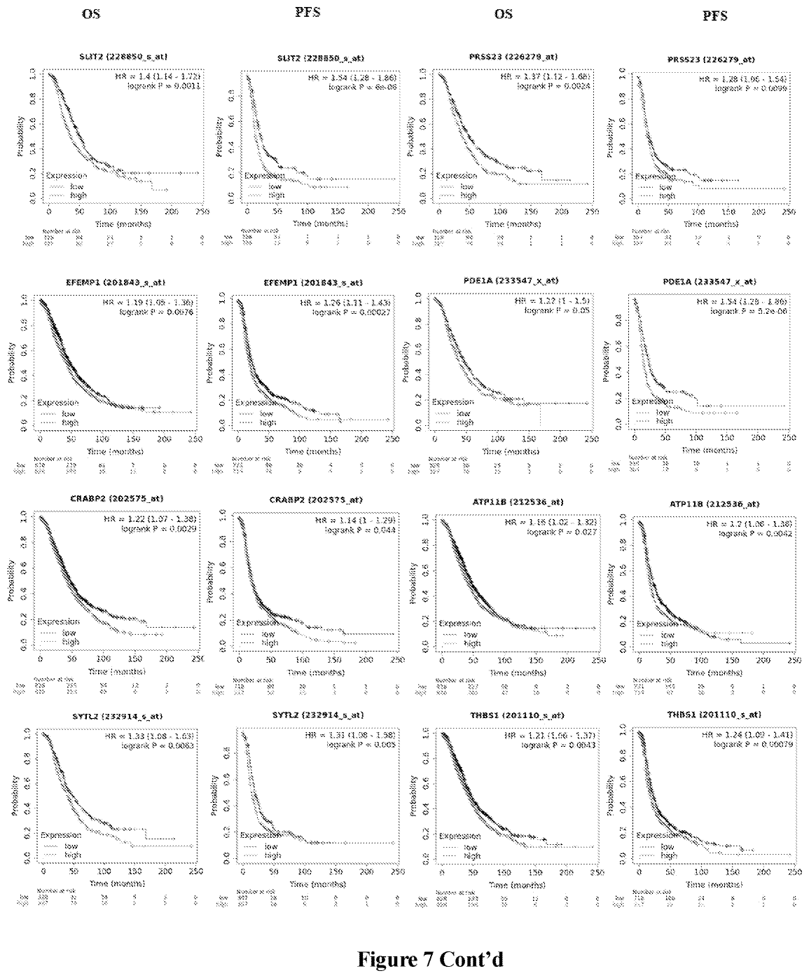

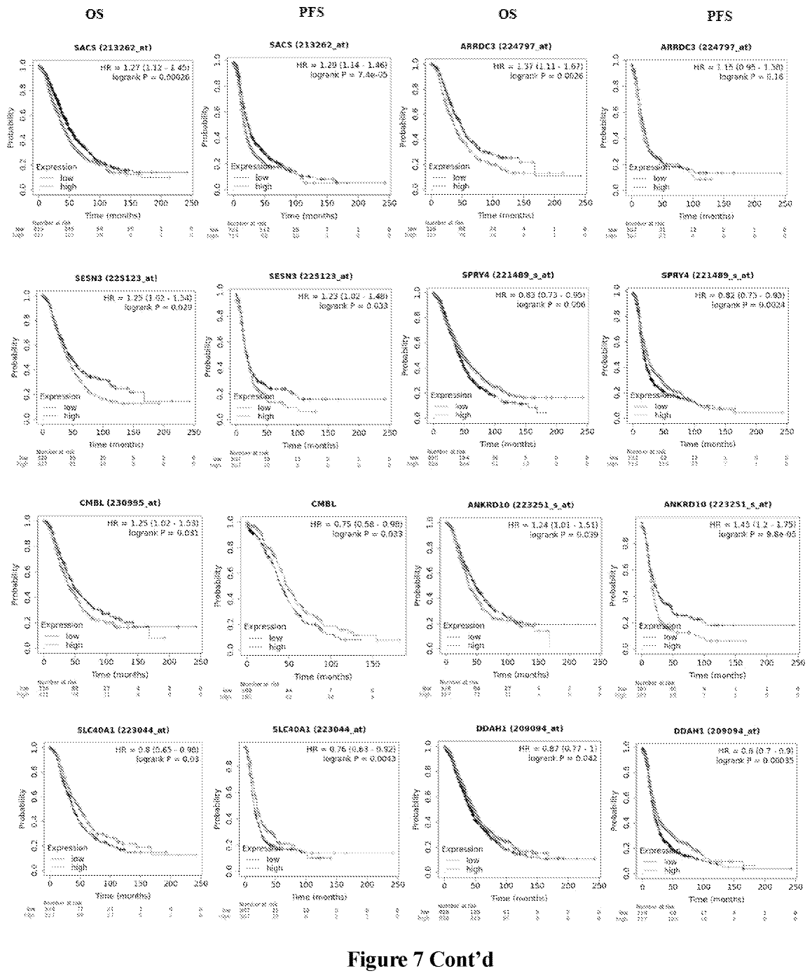

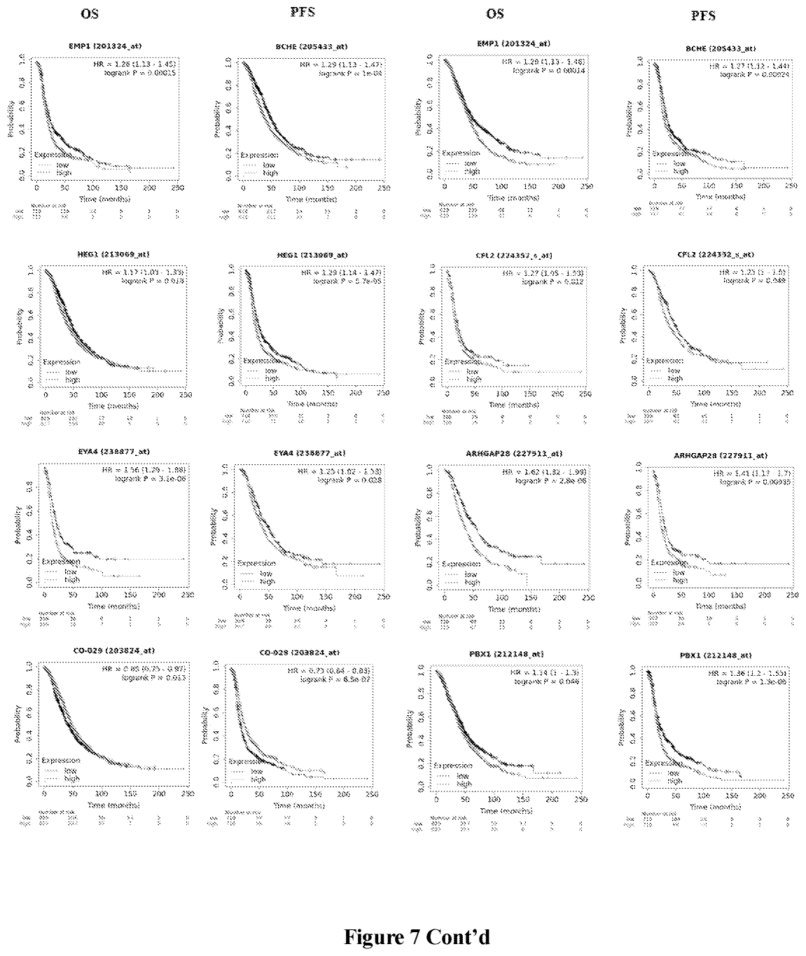

The top network in the list includes genes involved in survival pathways such as GAS1, VEGFC, KCNT2, and MAPK (FIG. 1D). The second network included SYTL2,ERK, ABCA3, and PRKG1, which are associated with molecule and vesicle trafficking, downstream phosphorylation, and cGMP signaling (FIG. 1E). To select potential clinically relevant genes in ovarian cancer, we interrogated the KM plotter searchable patient database. The KM plotter includes data from “The Cancer Genome Atlas” (TCGA) data portal and other patient databases for a total of 1,656 ovarian cancer samples. The correlation between the 414 genes and the Overall Survival (OS) and the Progression-Free Survival was assessed. Using the KM plotter database, 61 genes showed a difference in the OS and/or PFS. FIG. 2A shows the Kaplan-Meier curves for the top four relevant genes (CASC10, PDLIM3, EMP1, and ATP11B) of the list. A strong correlation between the RNA expression levels and the OS and the PFS was observed for the four genes shown in FIG. 2A(a)-2A(d). The Kaplan-Meier curves for the other 57 genes are shown in FIG. 7. The differential expression levels of the 61 genes were validated by real-time PCR. As shown in Table 4, 45 out of the 61 genes were validated by PCR, 28 were upregulated, and 17 were downregulated in OVCAR3CIS compared to its sensitive counterpart (FIG. 2B).

TABLE 4
Relative expression values of the 45 genes differentially
abundant in OVCAR3CIS vs. OVCAR3 cells.

| | qPCR | RNA-seq |
|---|---|---|
| GENE | Log2 FC | Log2FC |
| PDLIM3 | 13.16 | 5.56 |
| TPH2 | 11.53 | 8.04 |
| TRHDE | 10.67 | 7.82 |
| KONT2 | 10.47 | 5.09 |
| GAS1 | 10.24 | 5.01 |
| CLDN16 | 9.33 | 5.29 |
| PRRX1 | 9.09 | 4.31 |
| SESN3 | 9.07 | 3.41 |
| GALNT13 | 8.50 | 4.78 |
| SYTL2 | 7.63 | 4.55 |
| SULF1 | 7.43 | 3.78 |
| BCHE | 6.50 | 1.31 |
| ATP11B | 6.44 | 3.24 |
| SLIT2 | 5.33 | 2.09 |
| SLC6A15 | 5.29 | 5.45 |
| SH2D5 | 5.19 | 2.51 |
| VEGFC | 4.85 | 4.84 |
| CFL2 | 4.42 | 1.98 |
| ARRDC3 | 4.17 | 2.24 |
| CRABP2 | 3.43 | 1.62 |
| NUDT11 | 3.34 | 2.13 |
| EYA4 | 2.74 | 0.58 |
| SACS | 2.55 | 1.03 |
| KIF1A | 2.36 | 1.43 |
| ARHGAP28 | 2.19 | 1.24 |
| EMP1 | 2.11 | 1.20 |
| DDAH1 | 2.09 | 0.96 |
| CASC10 | 2.06 | 0.50 |
| LRRC17 | −0.94 | −1.42 |
| LRG1 | −0.97 | −2.72 |
| TMCC3 | −1.03 | −2.25 |
| SPRY4 | −1.67 | −1.91 |
| F2R | −2.09 | −3.80 |
| SULT1A1 | −2.67 | −3.43 |
| PBX1 | −2.78 | −2.29 |
| SLC7A2 | −2.85 | −5.47 |
| MXRA8 | −3.16 | −4.14 |
| NID1 | −3.36 | −4.50 |
| RUNX1 | −3.59 | −1.64 |
| PROCR | −4.28 | −5.77 |
| LIPG | −5.20 | −5.68 |
| PDE1A | −5.76 | −7.53 |
| PTPRD | −6.38 | −7.88 |
| VCAN | −6.60 | −5.64 |
| THBS1 | −9.79 | −4.06 |

RNAi screening was then performed by transiently transfecting the OVCAR3CIS cells with a pool of four specific siRNAs against each of the 27 genes, followed by colony formation assays (FIG. 2C). A greater than 50% reductions in the number of colonies for CASC10, ATP11B, EMP1, GAS1, SLC6A15, GALNT13, and PDLIM3 compared with cells transfected with a NC-siRNA (FIG. 2C).

### CASC10 is Upregulated in Ovarian Cancer Patients and Cisplatin-Resistant Ovarian Cancer Cells

According to survival analysis, CASC10 showed the strongest significant correlation between the OS (p =1.6e-09 HR =1.78) and PFS (p=1.5e-05 HR=1.57) of the disease (FIG. 2A)(a). A comparative expression (tumor vs. normal tissue) plot using the Gene Expression Profiling Interactive Analysis (GEPIA) searchable database (RNAseq data) revealed a statistically significant higher CASC10 expression in ovarian tumors compared to normal ovaries (FIG. 2D).

The expression of CASC10 was confirmed by real-time PCR in a panel of ovarian cancer cell lines. The CASC10 levels were higher in the cisplatin-resistant compared with the cisplatin-sensitive cells (FIG. 2E).

### CASC10 siRNA-Mediated Knockdown Reduced Cell Growth, Invasion, and Viability in Ovarian Cancer Cells

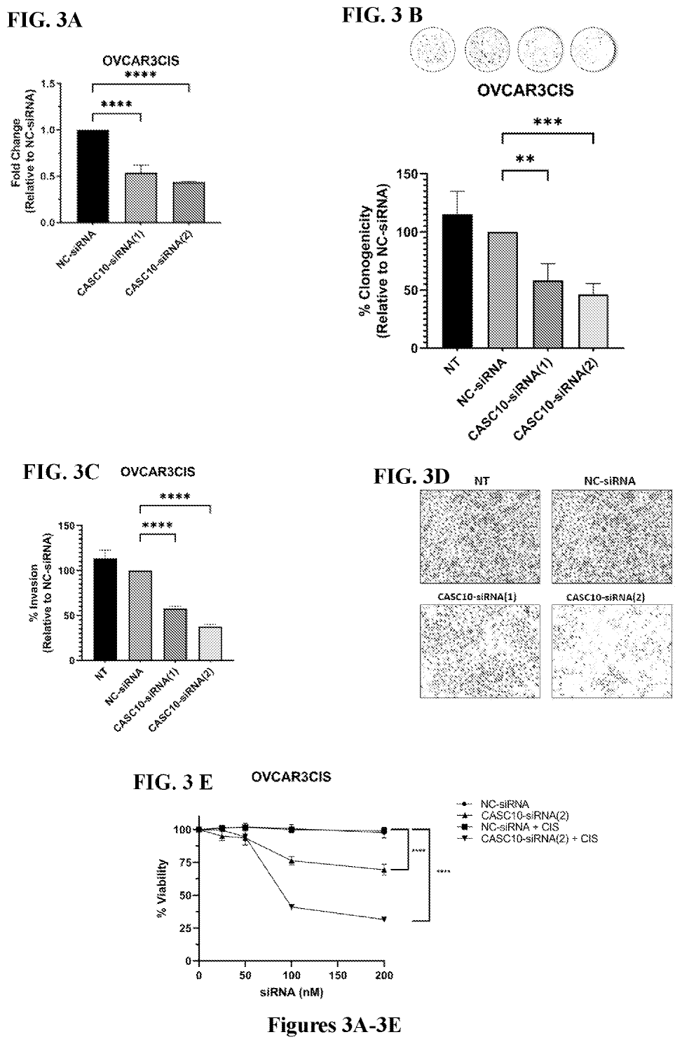

The biological consequences of siRNA mediated CASC 10 silencing in ovarian cancer cells were then studied. The 2-^{ΔΔcl} analysis of an RT-qPCR experiment showed that transient transfection of OVCAR3CIS cells with CASC 10-targeted siRNAs decreased the CASC10 expression by 47% with the CASC10-siRNA (1) and in 57% with CASC10-siRNA (2) as compared with the NC-siRNA (^{***}p<0.001, FIG. 3A). In a colony formation assay with OVCAR3CIS, both CASC 10-targeted siRNAs reduced the number of colonies formed compared with NC-siRNA transfected cells (FIG. 3B). Notably, the CASC10-siRNA (2) reduced the number of OVCAR3CIS colonies by 54% (^{***}p<0.0001), whereas CASC10-siRNA (1) reduced the number of colonies by only 42% (^{**}p<0.001). The effect of CASC10 knockdown was assessed on the invasion ability of OVCAR3CIS cells. Invasion assays showed that CASC10-siRNA (1) and CASC10-siRNA (2) significantly reduced the invasiveness of OVCAR3CIS (42% reduction; (^{***}*p<0.0001 and 62% reduction; ^{****}p<0.0001, respectively) compared with NC-siRNA transfected cells (FIG. 3C-D).

In order to assess the effects of CASC10 knockdown in a different type of OC cells other than the clear cell ovarian carcinoma cell line HGSOC SKOV3ip1 was used.

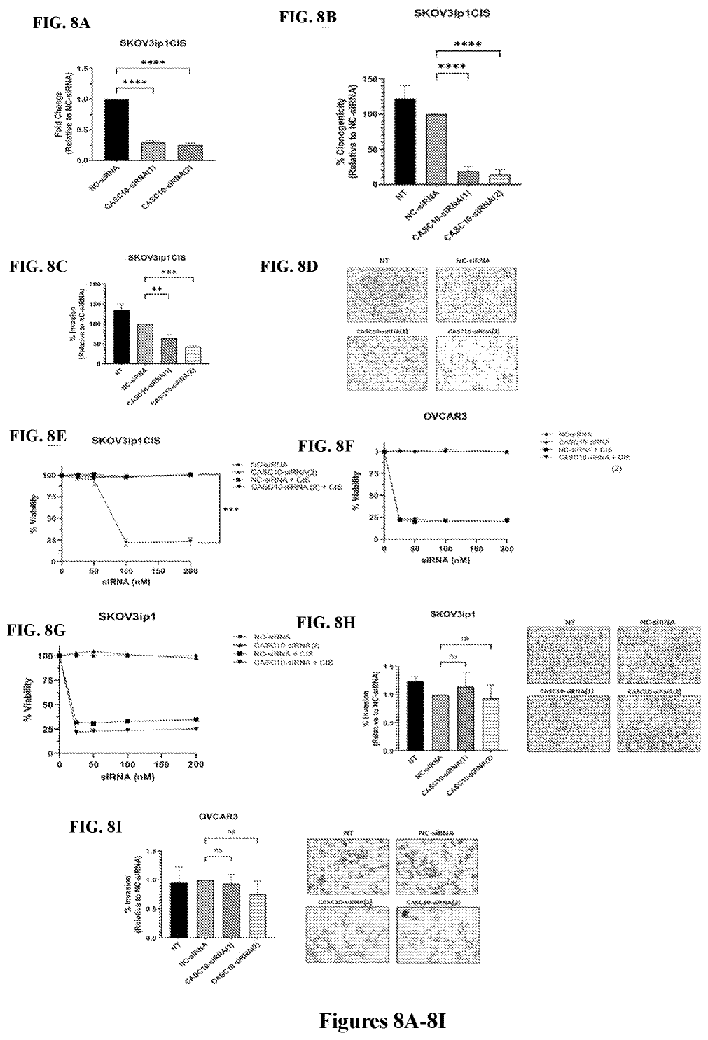

The 2-^{ΔΔCt} analysis showed a decrease of CASC10 relative expression of 70% (p<0.0001) and 75% (^{****}p<0.0001) following transfection of SKOV3ip1 cells with CASC10-SIRNA (1) and CASC10-siRNA (2) respectively (FIG. 8A). Surprisingly, CASC10-siRNA (2) reduced the number of colonies by 86% (^{****}p<0.0001), whereas CASC10-siRNA (1) reduced the number of colonies by 81% (^{****}p<0.0001) (FIG. 8B).

CASC10 knockdown in SKOV3ip1CIS reduced the invasion ability of these cells by 36% (**p<0.01), and 58% (***p<0.0001) with the CASC10-siRNA (1) and CASC10-SiRNA (2), respectively (FIG. 8C-D).

Reduced cell viability was assessed in CASC10-targeted siRNAs alone or in combination with CIS. The NC-siRNA did not reduce the cell viability of OVCAR3CIS cells at any of the assessed concentrations (FIG. 2E). CIS (2.5 μM final concentration) reduced the cell viability of NC-siRNA-transfected cells. Transient transfections of 50 nM and 100 nM (Final concentrations) of CASC10-siRNA (2) into OVCAR3CIS significantly reduced (15% with 50 nM ^{**}p<0.001 and 30% with 100 nM, ^{**}p<0.001) cell viability compared with the NC-SiRNA (FIG. 3E). Surprisingly, the combination of CASC10-sIRNA (2) with CIS (2.5 μM) significantly reduced to 56% (^{**}p<0.0001) the cell viability compared with NC-siRNA (FIG. 3E). Similar cell viability results were obtained combined CASC10-targeted siRNA plus CIS in SKOV3ip1CIS cells (FIG. 8E). We also performed cell viability experiments combining CASC10-targeted siRNA in OVCAR3 and SKOV3ip1 cells. The CASC10-targeted siRNA (2) did not significantly reduce cell viability at any siRNA concentrations tested compared with the NC-SIRNA (FIG. 8F-G).

### CASC10 siRNA-Mediated Knockdown Induced Apoptosis and Cell Cycle Arrest

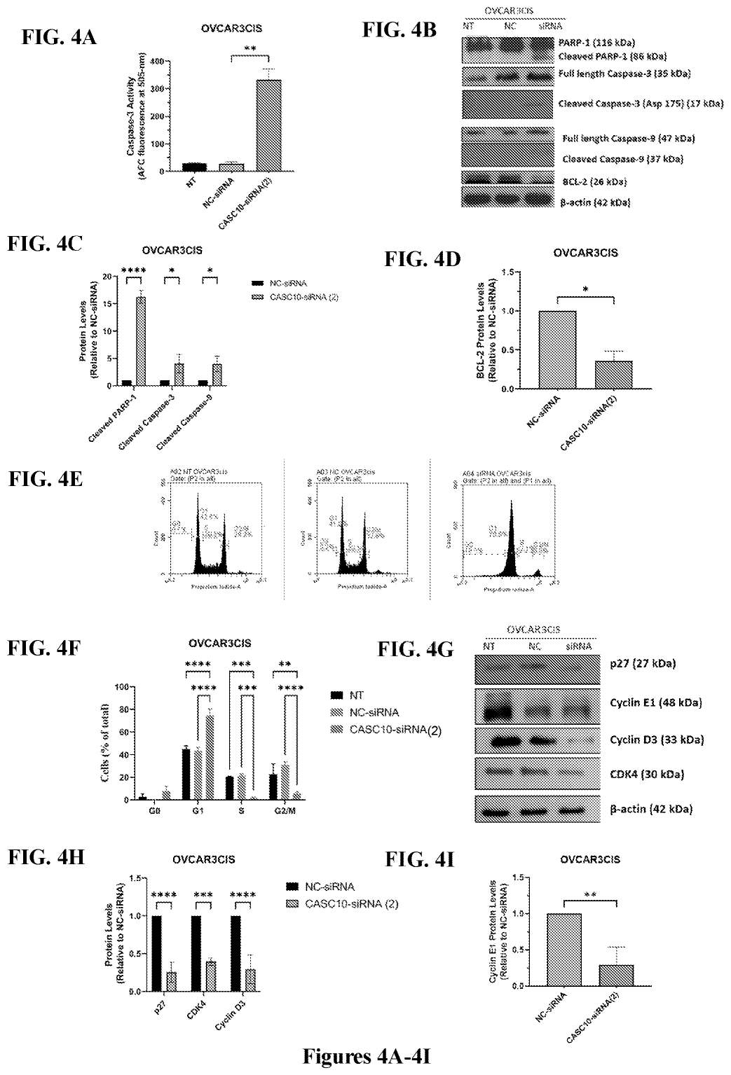

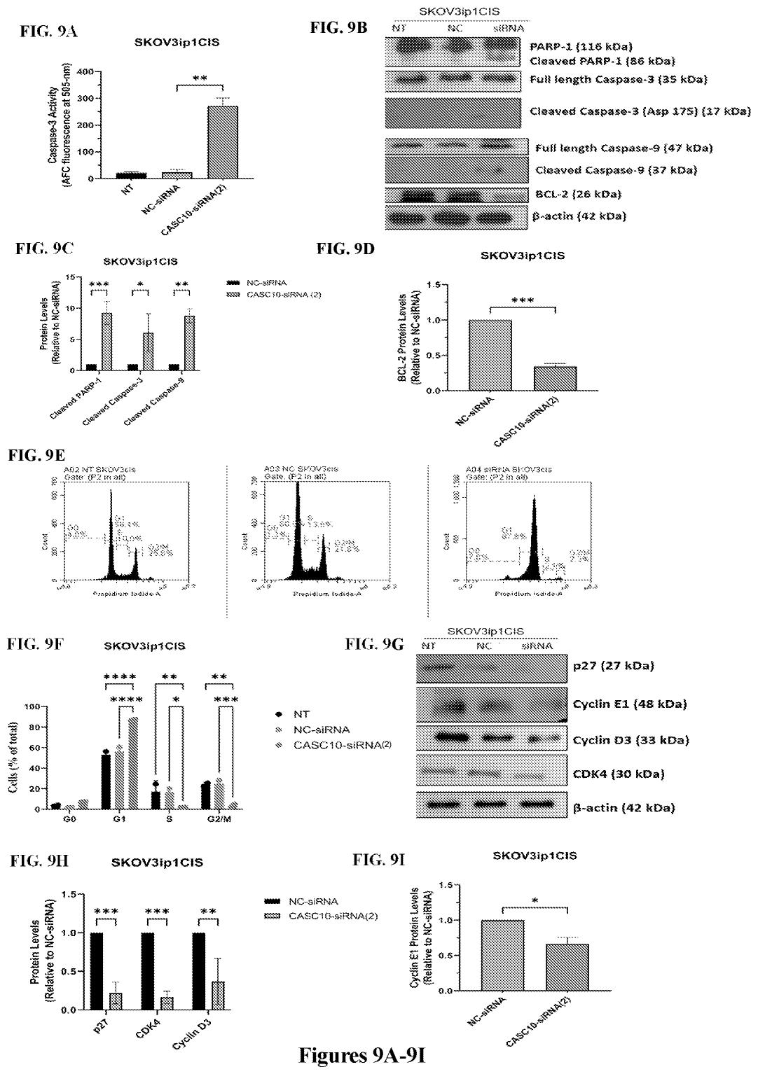

The reduction in cell growth and proliferation after CASC10 knockdown was assessed as related to activation of apoptosis, cell cycle arrest, or both. Compared to NC-SIRNA, SiRNA-mediated CASC10 knockdown in OVCAR3CIS cells resulted in a 5-fold increase in caspase-3 activity (^{**}p=0.0016. FIG. 4A). Similar results were obtained for SKOV3ip1CIS (4-fold increase; ^{**}p21 0.0016, FIG. 9A). Activation of apoptosis was confirmed by assessment of the changes in apoptotic-related proteins by western blot analysis. Cells treated with CASC10-siRNA (2) showed a significant increase in the active form of Caspase-9 (cleaved Caspase-9) and Caspase-3 (cleaved Caspase-3) (^{**}p<0.01 and ^{**}p<0.01, respectively, FIG. 4B-C). A significant increase in the cleaved poly-ADP ribose polymerase-1 (PARP-1) was also observed in CASC10-siRNA (2) as compared with NC-SiRNA transfected cells (^{**}p<0.001, FIG. 4C). Moreover, we observed a strong reduction of the anti-apoptotic protein, Bol-2, following siRNA-mediated siRNA knockdown compared with NC-siRNA-transfected cells (^{**}p=0.022, FIG. 4D). Similar results were observed for SKOV3ip1CIS (2-fold cleaved Caspase-9 increase: ^{***}p=0.0005, 6-fold cleaved Caspase-3 increase; ^{**}p=0.0076, and 7-fold cleaved PARP-1 increase: ^{****}p<0.0001, 66% Bcl-2 decrease; ^{***}p=0.0003) (FIG. 9B-D).

The effect of siRNA mediated CASC10 knockdown on cell cycle progression was assessed by flow cytometry. A dramatic and surprising cell cycle arrest in the G0/G1 to S phase was observed in OVCAR3CIS and SKOV3ip1CIS, 48 hours post-transfection (^{***}*p<0.0001, and ^{****}p<0.0001 respectively, FIG. 4E, F, and FIG. 9E-F). These results were confirmed by western blot, where we observed changes in key proteins involved in the G0/G1 to S phase checkpoint. Intriguingly, a reduction in the protein levels of the tumor suppressor p27 was observed in OVCAR3CIS-CASC10-SIRNA (2) and SKOV3ip1CIS-CASC10-siRNA (2) compared with NC-siRNA-transfected cells (^{****}p<0.0001, and ^{***}p=0.0002 respectively, FIG. 4G-I and FIG. 9G-I). In addition, a reduction of the checkpoint proteins of the S phase Cyclin E1, and CDK4 was observed in OVCAR3CIS-CASC10-siRNA (20 and SKOV3ip1CIS-CASC10-siRNA (2) compared with NC-siRNA-transfected cells (^{**}p=0.0017.^{*}p<0.03 and ***p=0.0001, ^{***}p=0.0007 respectively), FIG. 4G-I and FIG. 9G-I).

### In Vivo Targeting of CASC10 with Liposome-Encapsulated siRNAs

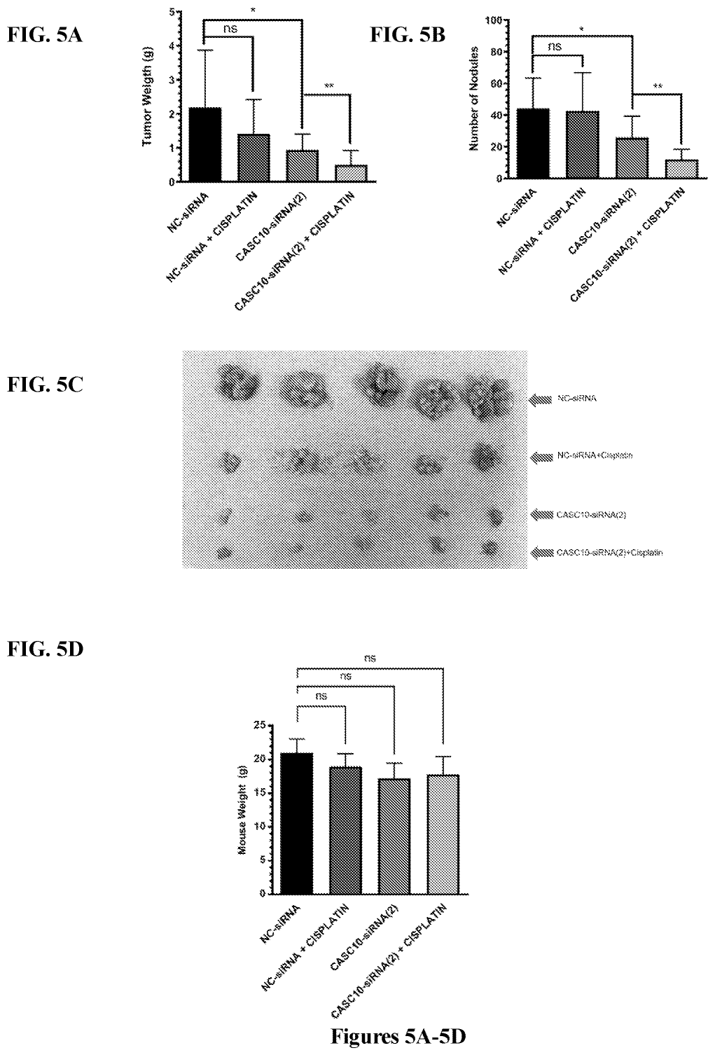

Next, siRNA-mediated CASC10 knockdown and reductions in in vivo tumor were assessed using encapsulated siRNAs into DOPC-based nanoliposomes. Tumor weight and nodule number were reduced in CASC10-siRNA group compared with NC-siRNA or cisplatin groups (^{*}p<0.05, FIG. 5A). The effects were exacerbated when CASC10-siRNA was combined with cisplatin (^{**}p<0.008 FIG. 5A-B). Weight differences among the different groups of mice were not observed at the end of the experiment (FIG. 5C). In summary, combination therapy of liposomal CASC10-siRNA and CIS attenuated tumor progression in a cisplatin-resistant mouse model of HGSOC.

### Downstream Effectors of CASC10 in HGSOC Cells

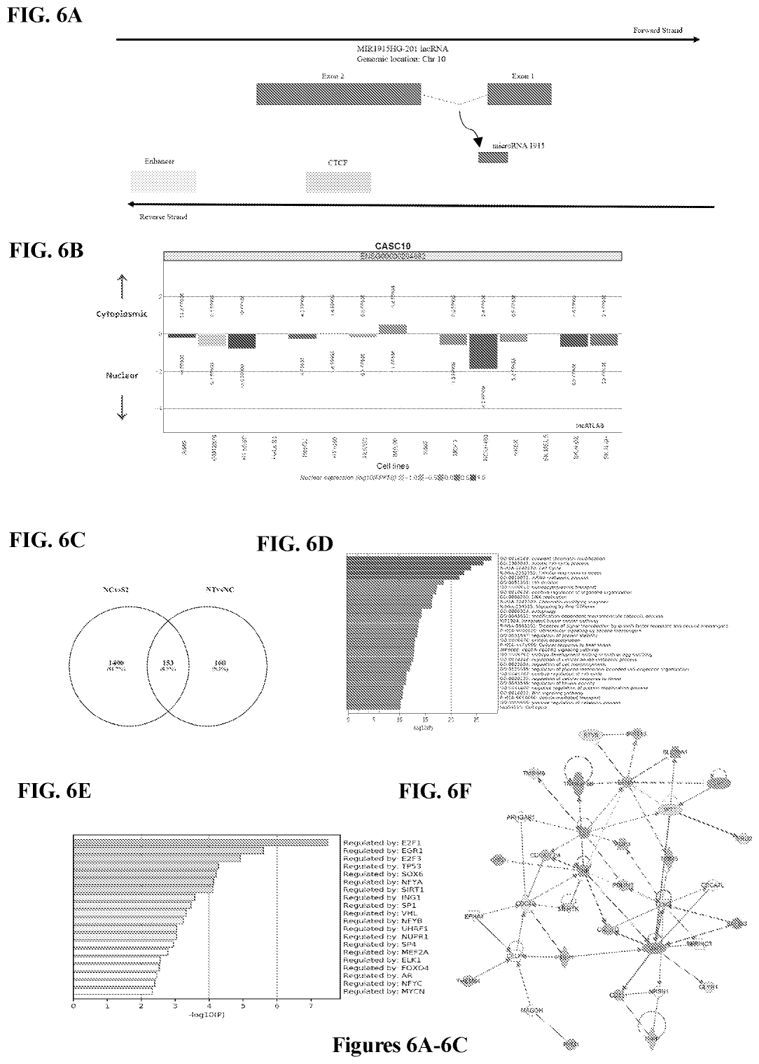

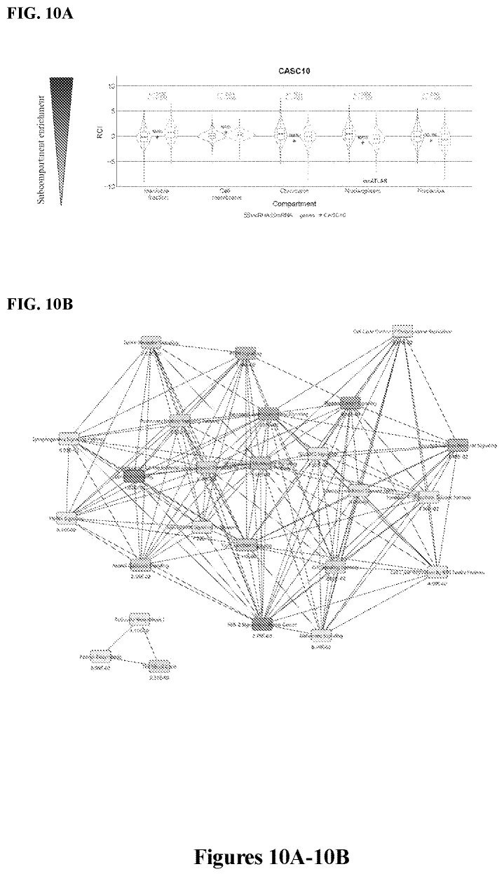

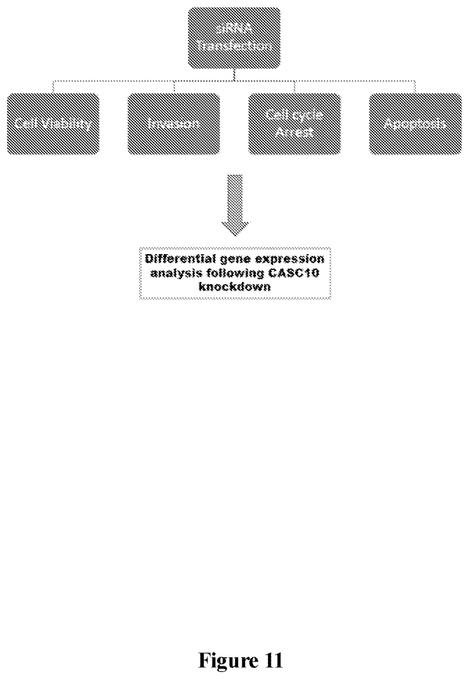

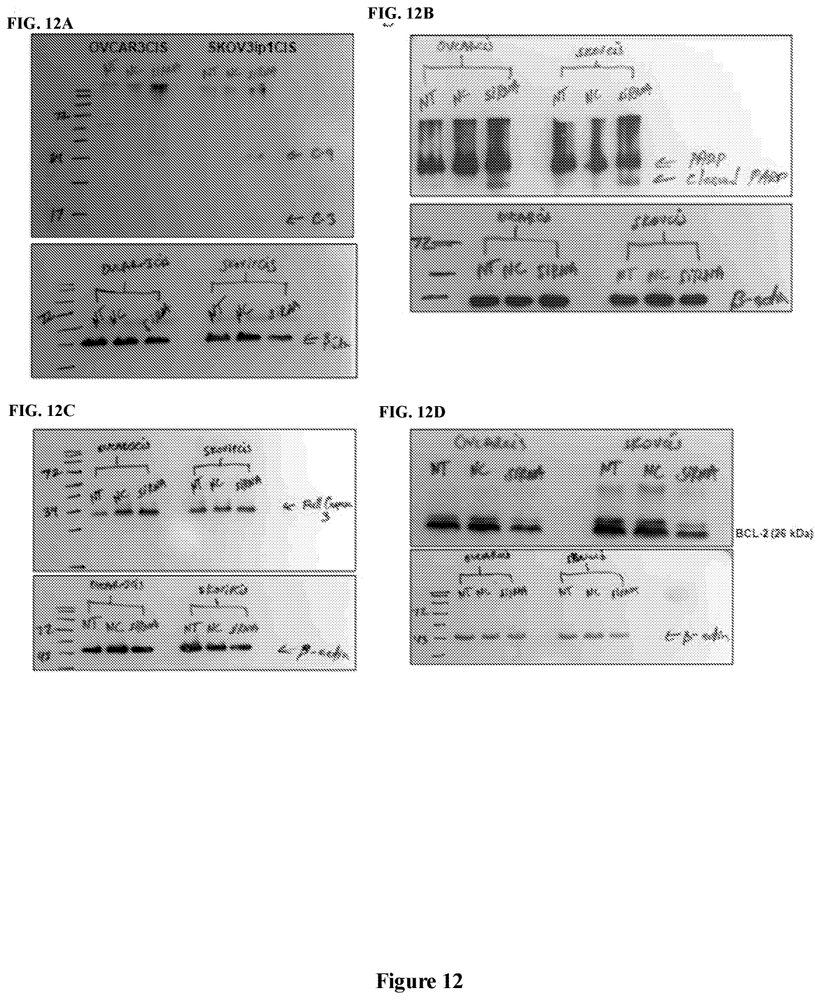

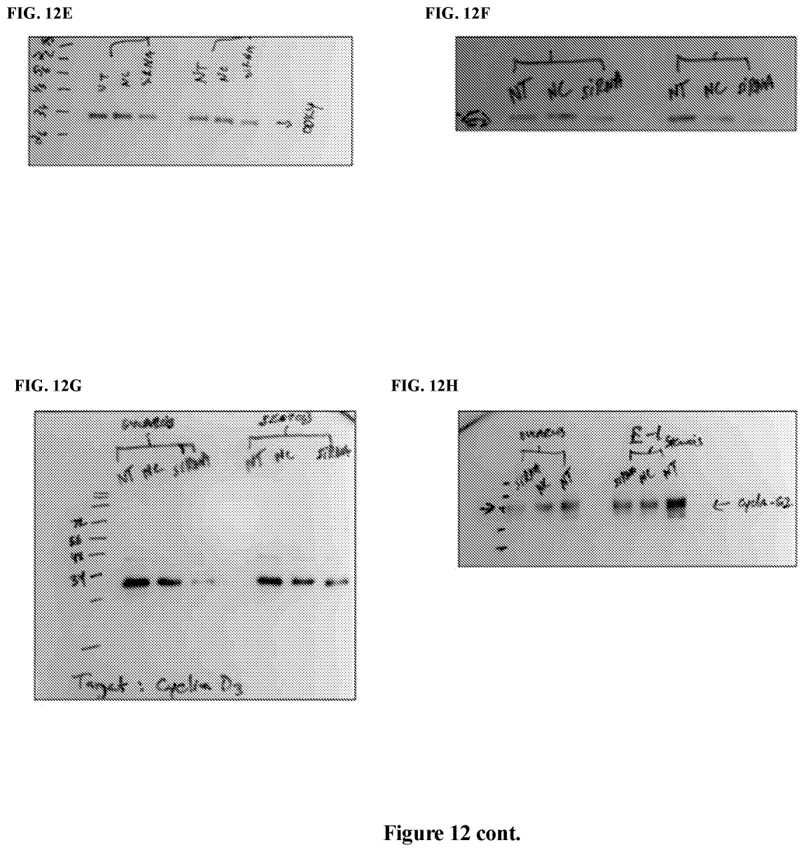

CASC10 is a long noncoding RNA (antisense lincRNA) with a length of 3,799 bp located in the reverse strand of chromosome 10 (FIG. 6A). Splicing of the transcribed RNA produces a 3,799 bp by elimination of an intronic region of 804 bp. Neither the biological role nor the cellular localization of this non-coding RNA is currently known. We used the LncATLAS, a web-based cell visualization tool that uses available subc-RNAseq raw data from 15 well-known cell lines from the ENCODE consortium and quantifies the RNA localization using the “relative concentration index” (RCI). RCI is defined as the log₂-transformed ratio of FPKM (fragments per kilobase per million mapped) in two samples (i.e., nucleus and cytoplasm). Results demonstrated that CASC10 expression is higher in the nuclear fraction than the cytoplasmic fraction in 10 out of the 15 well-known cell lines (FIG. 6B). In addition, enrichment of CASC10 RNA levels was observed in the chromatin sub compartment in the nucleus of K562 cells (FIG. 10A).

The signaling pathways downstream of CASC10, were further assessed using a transcriptome-wise analysis by RNA sequencing (RNA-seq) after siRNA-mediated CASC10 knockdown in OVCAR3CIS cells. Using an initial p-adjusted value (padj) cutoff <0.01, 1,560 differently abundant transcripts were identified between NC-siRNA and CASC10-siRNA (2). One hundred sixty transcripts were regulated in NC-siRNA as compared with non-treated cells, (see the Venn diagram, FIG. 6C). In total, 1,400 differentially abundant transcripts were exclusive of CASC10-siRNA (2) compared with NC-siRNA, including 736 downregulated and 816 upregulated transcripts.

The 1,400 differentially expressed genes (DEGs) of these transcripts were used to analyze functional enrichment using Metascape via Gene Ontology (GO) and the Kyoto

Encyclopedia of Genes and Genomes (KEGG). The top 20 most significantly (p-value ≤0.01) enriched ontology clusters include mitotic cell cycle processes, histone modifications, cell cycle, mRNA metabolic processes, cellular response to stress, and cellular response to DNA damage stimulus (FIG. 6D). In addition, Metascape was used to identify transcriptional regulatory transcription factors (TFs) for the identified DEGs. Most enriched ontology clusters were regulated by transcription factors such as E2F1, EGR1, E2F3, TP53,SOX6, NFYA, and SIRT1 (FIG. 6E). A further log2 fold change cutoff >1.2 or <−1.2 with a p-value ≤0.01 was used to select the most relevant differentially expressed genes following CACS10 knockdown. Applying these criteria, 32 differentially expressed genes were identified, 18 upregulated and 14 downregulated in CASC10-siRNA (2) vs. NC-siRNA transfected cells. Based on these criteria, among the upregulated genes, the top five included RTN4R, KIAA0754, PYM1, CNN1, and TGFBRAP1 (Table 5). The top five of the 14 downregulated genes include NUP43, FHL1, DHFR2, MIR1915HG, and NDUFA7 (Table 5).

TABLE 5
Top five upregulated and top five downregulated genes in
CASC10-siRNA(2) vs. NC-siRNA

| | | | |
|---|---|---|---|
| Gene Symbol | Gene Name | Biological Role | p-value |

Upregulated

| | | | |
|---|---|---|---|
| RTN4R | Reticulon 4 receptor | Receptor mediated | 4.59E−21 |
| | | axonal growth | |
| | | inhibition | |
| MACF1 | Microtubule actin | Actin-microtubule | 2.83E−12 |
| | crosslinking factor 1 | interactions | |
| PYM1 | PYM homolog 1 | Nuclear-transcribed | 5.24E−20 |
| | | mRNA catabolic | |
| | | process and | |
| | | regulation of translation | |
| CNN1 | Cellular | Regulation of | 7.38E−06 |
| | communication | vascular associated | |
| | network factor 1 | smooth muscle | |
| | | cell proliferation | |
| TGFBRAP1 | orming growth | TGF-beta signaling | 3.32E−39 |
| | factor beta receptor | and association | |
| | associated protein 1 | to SMAD4 | |

Downregulated

| | | | |
|---|---|---|---|
| NUP43 | Nucleoporin 43 | Transport of | 1.69E−86 |
| | | macromolecules | |
| | | between cytoplasm | |
| | | and nucleus | |
| FHL1 | Four and a | Assembly of sarcomeres | 2.49E−11 |
| | half LIM | and muscle | |
| | domains 1 | contraction regulation | |
| DHFR2 | Dihydrofolate | Tetrahydrofolate | 1.21E−10 |
| | reductase 2 | metabolic process | |
| | | and thymidine | |
| | | biosynthesis | |
| MIR1915HG | microRNA1915 | Long Non-coding RNA | 2.76E−59 |
| | host gene | | |
| NDUFA7 | NADH Ubiquinone | Member of complex | 7.76E−29 |
| | Oxidoreductase | I in the electron | |
| | Subunit | transport chain | |
| | A7 | | |

IPA was performed to better visualize the molecular interactions between the 32 differentially abundant transcripts. The top network in the list includes genes involved in cell death and survival pathways such as Cyclin D, MERTK, TNF, and CDK4 (FIG. 6F). In addition, the top canonical pathways involved HER-2 signaling in breast cancer, cell cycle, regulation by BTG family proteins, cell cycle control of chromosomal replication, and PTEN signaling (FIG. 10B).

### Conclusion

RNAseq followed bioinformatics, OS, and PFS KM curves, and an RNAi screening identified several potential genes for ovarian cancer therapy. Particularly, siRNA-mediated knockdown of seven genes, CASC10, ATP11B, EMP1, GAS1, SLC6A15, GALNT13, and PDLIM3, significantly reduced cell proliferation of ovarian cancer cells. Elevated ATP11B levels promote the export of cisplatin from cells. CASC10, also known as MIR1915HG, is a IncRNA of unknown cellular localization and function. LncRNAs molecules play important roles at every step of the gene expression course, including regulation of transcription, posttranscriptional processing, genomic imprinting, chromatin modification, and regulation of protein function. Herein is disclosed the unexpected result that CASC10 is increased in ovarian cancer samples compared with control ovaries and in cisplatin-resistant ovarian cancer cells compared with cisplatin sensitive cells counterparts and that blocking CASC10 has beneficial effects in ovarian cancer. CASC10 belongs to the CASC family, and the results herein are unexpected as deregulation of other members of the CASC family (CASC2, CASC11, CASC9) are associated with enhanced proliferation of cancer cells. More surprising, was the cell cycle arrest, specifically in the GO/G1 to S phase transition following CASC10-siRNA knockdown. These results were confirmed by a reduction in the Cyclin E1 and CDK4 protein levels upon CASC10 knockdown. Further surprising were reduced protein levels of the cell cycle inhibitory protein p27 following CASC10 knockdown.

The conventional treatment for ovarian cancer includes the use of cisplatin and paclitaxel, to which many patients develop chemoresistance leading to a therapeutic Failure. The liposomal CASC-10 siRNA disclosed herein reduced tumor growth and metastasis in an HGSOC mouse model. This effect was exacerbated when the liposomal formulation was combined with cisplatin. In addition, several genes were regulated following CASC10 knockdown.

TABLE 6
Sequence Table - siRNA

| SEQ ID | | |
|---|---|---|
| NO. | SIRNA | Sequence |
| 1 | CASC10- | 5′ GCUAUCUGCUUGAUCCCUU(dT)(dT) 3′ |
| | SIRNA (1) | |
| 2 | | 5′ AAGGGAUCAAGCAGAUAGO(dT)(dT) 3′ |
| 3 | CASC10- | 5′ GACUCUUGGAUCCAAGUUU(dT)(dT) 3′ |
| | SIRNA (2) | |
| 4 | | 5′ AAACUUGGAUCCAAGAGUC(dT)(dT) 3′ |

### REFERENCES

[1] R. L. Siegel, K. D. Miller, H. E. Fuchs, and A. Jemal, “Cancer statistics, 2022.,” CA Cancer J Clin, vol. 72, no. 1, pp. 7-33, 2022.
[2] M. McMullen, K. Karakasis, R. Rottapel, and A. M. Oza, “Advances in ovarian cancer, from biology to treatment,” Nature Cancer, vol. 2, no. 1, pp. 6-8, 2021.
[3] M. A. Lisio, L. Fu, A. Goyeneche, Z. H. Gao, and C. Telleria, “High-grade serous ovarian cancer: Basic sciences, clinical and therapeutic standpoints,” International Journal of Molecular Sciences, vol. 20, no. 4, 2019.
[4] P. A. Vasey, “Resistance to chemotherapy in advanced ovarian cancer: Mechanisms and current strategies,” British Journal of Cancer, vol. 89, no. SUPPL. 3, pp. 23-28, 2003.
[5] B. Van Zyl, D. Tang, and N. A. Bowden, “Biomarkers of platinum resistance in ovarian cancer: What can we use to improve treatment,” Endocrine-Related Cancer, vol. 25, no. 5, pp. R303-R318, 2018.
[6] I. M. Echevarría-Vargas, F. Valiyeva, and P. E. Vivas-Mejia, “Upregulation of miR-21 in cisplatin resistant ovarian cancer via JNK-1/c-Jun pathway,” PLOS ONE, vol. 9, no. 5, 2014.
[7] P. M. Báez-Vega et al., “Targeting miR-21-3p inhibits proliferation and invasion of ovarian cancer cells,” Oncotarget, vol. 7, no. 24, pp. 36321-36337, 2016.
[8] B. Gyorffy, A. Lánczky, and Z. Szállási, “Implementing an online tool for genomewide validation of survival-associated biomarkers in ovarian-cancer using microarray data from 1287 patients,” Endocrine-Related Cancer, vol. 19, no. 2, pp. 197-208, 2012.
[9] Y. Santana-Rivera et al., “Reduced expression of enolase-1 correlates with high intracellular glucose levels and increased senescence in cisplatin-resistant ovarian cancer cells,” American Journal of Translational Research, vol. 12, no. 4, pp. 1275-1292, 2020.
[10] R. J. Parker, A. Eastman, F. BostickBruton, and E. Reed, “Acquired cisplatin resistance in human ovarian cancer cells is associated with enhanced repair of cisplatin-DNA lesions and reduced drug accumulation,” Journal of Clinical Investigation, vol. 87, no. 3, pp. 772-777, 1991.
[11] B. M. Barnes et al., “Distinct transcriptional programs stratify ovarian cancer cell lines into the five major histological subtypes,” Genome Medicine, vol. 13, no. 1, pp. 1-19, 2021.
[12] K. J. Livak and T. D. Schmittgen, “Analysis of relative gene expression data using real-time quantitative PCR and the 2-AACT method,” Methods, vol. 25, no. 4, pp. 402-408, 2001.
[13] J. M. Reyes-gonzález et al., “Targeting c-MYC in platinum-resistant ovarian cancer,” vol.
   14, no. 10, pp. 2260-2269, 2016.
S. E. Dobbins et al., “Europe PMC Funders Group Common variation at 10p12. 31 near MLLT10 influences meningioma risk,” vol. 43, no. 9, pp. 825-827, 2016.
[15] D. Mas-Ponte, J. Carlevaro-Fita, E. Palumbo, T. H. Pulido, R. Guigo, and R. Johnson, “LncATLAS database for subcellular localization of long noncoding RNAs,” Rna, vol. 23, no. 7, pp. 1080-1087, 2017.
[16] Y. Zhou et al., “Metascape provides a biologist-oriented resource for the analysis of systems-level datasets,” Nature Communications, vol. 10, no. 1, 2019.
[17] M. Moreno-Smith et al., “ATP11B mediates platinum resistance in ovarian cancer,” J Clin Invest, vol. 128, no. 7, p. 3199, 2018 .
[18] Y. Liu, Y. Ding, Y. Nie, and M. Yang, “EMP1 promotes the proliferation and invasion of ovarian cancer cells through activating the MAPK pathway,” Onco Targets and Therapy, vol. 13, pp. 2047-2055, 2020.
[19] E. Bignotti et al., “Gene expression profile of ovarian serous papillary carcinomas: identification of metastasis-associated genes.” American Journal of Obstetrics and Gynecology, vol. 196, no. 3, pp. 245.e1-245.e11, 2007.
[20] A. Necsulea et al., “The evolution of IncRNA repertoires and expression pattems in tetrapods,” Nature, vol. 505, no. 7485, pp. 635-640, 2014 .
[21] T. Derrien et al., “The GENCODE v7 catalog of human long noncoding RNAs: Analysis of their gene structure, evolution, and expression,” Genome Research, vol. 22, no. 9, pp. 1775-1789, 2012.
[22] S. Katayama et al., “Molecular biology: Antisense transcription in the mammalian transcriptome,” Science (1979), vol. 309, no. 5740, pp. 1564-1566, 2005.
[23] I. Ulitsky and D. P. Bartel, “XLincRNAs: Genomics, evolution, and mechanisms,” Cell, vol. 154, no. 1, p. 26, 2013.
[24] E. Kelemen, J. Danis, A. Göblös, Z. Bata-Csörgö, and M. Széll, “Exosomal long non-coding RNAs as biomarkers in human diseases,” Electronic Journal of the International Federation of Clinical Chemistry and Laboratory Medicine, vol. 30, no. 2, pp. 224-236, 2019.
[25] T. Gutschner and S. Diederichs, “The hallmarks of cancer: A long non-coding RNA point of view.” RNA Biology, vol. 9, no. 6, pp. 703-719, 2012.
[26] Y. Shi, S. Gao, Y. Zheng. M. Yao, and F. Ruan, “Lncma casc15 functions as an unfavorable predictor of ovarian cancer prognosis and inhibits tumor progression through regulation of mir-221/arid1a axis,” OncoTargets and Therapy, vol. 12, pp. 8725-8736, 2019.
[27] T. He et al., “Long non-coding RNA CASC15 is upregulated in hepatocellular carcinoma and facilitates hepatocarcinogenesis,” International Journal of Oncology, vol. 51, no. 6, pp. 1722-1730 December 2017.
[28] S. Shan et al., “Higher IncRNA CASC15 expression predicts poor prognosis and associates with tumor growth in cervical cancer,” European Review for Medical and Pharmacological Sciences, vol. 23, no. 2, pp. 507-512, 2019.
[29] W. Hsu, L. Liu, X. Chen, Y. Zhang, and W. Zhu, “LncRNA CASC11 promotes the cervical cancer progression by activating Wnt/beta-catenin signaling pathway,” Biol Res, vol. 52, no. 1, p. 33, 2019.
[30] K. Luo et al., “LncRNA CASC9 interacts with CPSF3 to regulate TGF-β signaling in colorectal cancer,” Journal of Experimental and Clinical Cancer Research, vol. 38. no. 1, pp. 1-16, 2019.
[31] W. Dai et al., “Berberine promotes apoptosis of colorectal cancer via regulation of the long non-coding RNA (LncRNA) cancer susceptibility candidate 2 (CASC2)/AU-binding factor 1 (AUF1)/B-cell cll/lymphoma 2 (Bcl-2) axis,” Medical Science Monitor, vol. 25, pp. 730-738, 2019.
[32] M. Cuadrado, P. Gutierrez-Martinez, A. Swat, A. R. Nebreda, and O. Fernandez-Capetillo, “P27Kip1 stabilization is essential for the maintenance of cell cycle arrest in response to DNA damage,” Cancer Research, vol. 69, no. 22, pp. 8726-8732, 2009.
[33] K. Hiromura, J. W. Pippin, M. L. Fero, J. M. Roberts, and S. J. Shankland, “Modulation of apoptosis by the cyclin-dependent kinase inhibitor p27 (Kip1),” Journal of Clinical Investigation, vol. 103, no. 5, pp. 597-604, 1999.
[34] B. Levkau et al., “Cleavage of p21Cip1/Waf1 and p27Kip1 Mediates Apoptosis in Endothelial Cells through Activation of Cdk2: Role of a Caspase Cascade,” Cell, vol. 1, pp. 553-563, 1998.
[35] J. M. Reyes-González and P. E. Vivas-Mejía, “c-MYC and Epithelial Ovarian Cancer,” Frontiers in Oncology, vol. 11, no. February, pp. 1-7, 2021.
[36] R. W. Yao, Y. Wang, and L. L. Chen, “Cellular functions of long noncoding RNAS.”
   Nature Cell Biology, vol. 21, no. 5, pp. 542-551, 2019.
[37] C. Tian, S. Zhou, and C. Yi, “High NUP43 expression might independently predict poor overall survival in luminal A and in HER2+breast cancer,” Future Oncology, vol. 14, no. 15,pp. 1431-1442, 2018.
[38] B. M. A. Papia Chakraborty, Yaming Wang, Jen-Hsuan Wei, Jan van Deursen, Hongtao Yu, Liviu Malureanu, Mary Dasso, Douglass J. Forbe, David E. Levy, Joachim Seemann,, And Fontoura, “Nucleoporin levels regulate cell cycle progression and phase-specific gene expression,” Cmes-Computer Modeling in Engineering & Sciences, vol. 1, no. 2, pp. 119-131, 2000.
[39] X. Xu, J. Nie. L. Lu, C. Du, F. Meng, and D. Song, “LINC00337 promotes tumor angiogenesis in colorectal cancer by recruiting DNMT1, which suppresses the expression of CNN1.” Cancer Gene Therapy, vol. 28, no. 12, pp. 1285-1297, 2021.
[40] Z. Wang, T. E. Li, M. Chen, J. J. Pan, and K. W. Shen, “miR-106b-5p contributes to the lung metastasis of breast cancer via targeting CNN1 and regulating Rho/ROCK1 pathway,” Aging, vol. 12, no. 2, pp. 1867-1887, 2020.

What is claimed is:

1. A method of treating cancer in a subject in need thereof, comprising administering an siRNA against one or more target genes SACS, CASC10, EMP1, GAS1, SLC6A15, GALNT13, ATP11B, and PDLIM3, wherein the target gene expression is reduced following SIRNA administration.

2. The method of claim 1, wherein the siRNA is CASC10.

3. The method of claim 1, wherein the cancer is ovarian cancer.

4. The method of claim 1, wherein the siRNA is packaged inside a liposome.

5. The method of claim 1, wherein siRNA administration upregulates one or more of RTN4R, KIAA0754, PYM1, CNN1, and TGFBRAP1.

6. The method of claim 1, wherein siRNA administration downregulates one or more of NUP43, FHL1, DHFR2, MIR1915HG, and NDUFA7

7. A liposome for use in treating ovarian cancer wherein the liposome contains one or more of CASC10, SACS, EMP1, GAS1, SLC6A15, GALNT13, ATP11B, or PDLIM3 siRNA.

8. The liposome of claim 7, wherein the siRNA is CASC10.

9. A pharmaceutical composition comprising the liposome containing siRNA of claim 7 and a pharmaceutically acceptable carrier.

10. The pharmaceutical composition of claim 9, wherein the siRNA is CASC10.

11. A kit comprising the liposome containing an siRNA according to any one of claims 1-8.

12. A liposome formulation for in vivo delivery of one or more siRNA comprising an SiRNA mixed with:
DOPC in about a 1:2 to about a1: 20 ratio;
DSPE-PEG-2000 at a concentration of about 1% to about10% mol/mol of DOPC; and
cholesterol at a concentration of about 10% to about 40% w/w of DOPC.

13. The liposome formulation of claim 12, wherein the siRNA is one or more of CASC10, SACS, EMP1, GAS1, SLC6A15, GALNT13, ATP11B, or PDLIM3.

14. The liposome formulation of claim 12, wherein the targeted gene is CASC10.

15. The liposome formulation of claim 12, wherein the DSPE-PEGO-2000 concentration is about 5% mol/mol of DOPC.

16. The liposome formulation of claim 12, wherein the cholesterol concentration is 20%.

17. A method of reducing cancer cell proliferation and/or invasion in an individual having ovarian cancer, the method comprising administering one or more of CASC10, SACS, EMP1, GAS1, SLC6A15, GALNT13, ATP11B, or PDLIM3 siRNA.

18. The method of claim 17, wherein the siRNA targets CASC10.

19. The method of claim 17, wherein the ovarian cancer is high-grade serous ovarian cancer (HGSOC).

20. The method claim 17, where in the individual has VCAR or OVCAR3CIS positive cells.

21. The method of claim 17, wherein administration of CASC10 reduces the number of ovarian cancer positive cell colonies by ≥50%.

22. The method of claim 21, wherein the positive colonies are comprised of VCAR, OVCAR3CIS, and/or SKOV3ip1CIS cells.

23. The method of claim 17, wherein administration of CASC10 reduces ovarian cancer cell viability by ≥10%.

24. The method of claim 17, wherein administration of CASC10 induces apoptosis of cisplatin resistant cancer cells as assessed by increased capase-9 and capase-3 activity.

25. A method of reducing ovarian cancer tumor size by administering encapsulated SiRNAs into DOPC-based liposomes, wherein the siRNA targets one or more of CASC10, SACS, EMP1, GAS1, SLC6A15, GALNT13, ATP11B, or PDLIM3.

26. A method for treating an individual with cisplatin-resistant ovarian cancer comprising administering to the individual CASC10 siRNA encapsulated in a liposome.

27. The method of claim 26, wherein the ovarian cancer is high-grade serous ovarian cancer (HGSOC).

28. The method claim 26, where in the ovarian cancer is comprised of VCAR, OVCAR3CIS, and/or SKOV3ip1CIS positive cells.


SEQUENCE LISTING (4 sequences)

SEQ ID NO: 1 - RNA, 21 nt, synthetic construct
  source 1..21: mol_type = other RNA
gctatctgcttgatcccttnn

SEQ ID NO: 2 - RNA, 21 nt, synthetic construct
  source 1..21: mol_type = other RNA
aagggatcaagcagatagcnn

SEQ ID NO: 3 - RNA, 21 nt, synthetic construct
  source 1..21: mol_type = other RNA
gactcttggatccaagtttnn

SEQ ID NO: 4 - RNA, 21 nt, synthetic construct
  source 1..21: mol_type = other RNA
aaacttggatccaagagtcnn